(19) United States
(12) Patent Application Publication
(10) Pub. No.: US 2012/0190800 A1
(43) Pub. Date: Jul. 26, 2012

(54) FUNCTIONALIZED POLYETHYLENE TEREPHTHALATE POLYMERS, FUNCTIONALIZED DERIVATIVE POLYMERS OF POLYETHYLENE TEREPHTHALATE, METHODS OF MAKING AND USING SAME

(76) Inventors: Kristopher M. Felice, Wolverine Lake, MI (US); George F. Willard, Ravenswood, WV (US); Eric W. Uffman, Maryland Heights, MO (US); Albert C. Lee, Creve Coeur, MO (US)

(21) Appl. No.: 13/190,066

(22) Filed: Jul. 25, 2011

Related U.S. Application Data
(60) Provisional application No. 61/404,621, filed on Oct. 6, 2010.
(60) Provisional application No. 61/444,998, filed on Feb. 21, 2011.

(51) Int. Cl. C08G 63/91; C07C 69/78

(52) U.S. Cl. 525/419; 560/85

(57) ABSTRACT

The presently disclosed and claimed inventive concept(s) relates generally to the functionalized polyethylene terephthalate polymers and functionalized derivatives of PET. More particularly, but not to be construed as limiting, the presently disclosed and claimed inventive concept(s) relate to lower molecular weight functionalized digested PET materials from digesting recycled and methods of making and using same.

### CROSS REFERENCE TO RELATED APPLICATIONS

The present application claims the benefit under 35 U.S.C. 119(e) of U.S. Provisional Application Ser. No. 61/404,621, filed Oct. 6, 2010, the entirety of which is hereby expressly incorporated herein by reference. The present application also claims the benefit under 35 U.S.C. 119(e) of U.S. Provisional Application Ser. No. 61/444,998, filed Feb. 21, 2011, the entirety of which is hereby expressly incorporated herein by reference.

### BACKGROUND OF THE INVENTION

1. Field of the Disclosed and Claimed Inventive Concepts

The presently disclosed and claimed inventive concept(s) relates generally to functionalized polyethylene terephthalate (“PET”) polymers and functionalized derivatives of PET (“fPET”). More particularly, but not to be construed as limiting, the presently disclosed and claimed inventive concept(s) relate to lower molecular weight functionalized digested PET materials (“dfPET”) made from digesting polyethylene terephthalate, especially recycled polyethylene terephthalate (“rPET”). In one particular aspect, the presently disclosed and claimed inventive concept(s) relate to the production of an oligomeric form of functionalized digested polyethylene terephthalic acid from waste products, such as beverage containers, made from polyethylene terephthalate. In one embodiment, the dfPET polymers have a MW of from about 200 to about 2000. These dfPET polymers have excellent solubility in various organic solvents and provide a functionalized backbone for the production of polymeric based products such as polyurethane dispersions (PUDs) and polyurethane resins (PURs), by way of example but not by way of limitation.

2. Background

Most polyester resins used in commercial applications are formed from raw materials which are rising in price and have relatively large markets. Accordingly, recovery of these raw materials from scrap, waste and used products is an important economical consideration as well as an ecological consideration. One widely used polyester is polyethylene terephthalate (hereinafter “PET”) made from terephthalic acid and ethylene glycol. Additionally, a Bisphenol A polyester resin could be used in a manner similar to PET.

Over the past 20 years, there has been an increased push throughout the world to increase recycling of polyester resins. Plastic bottles commonly used for drinks and carbonated beverages, for example, are made from polyethylene terephthalate and represent a large potential source of recoverable polyesters: either as bulk refined PET or the terephthalic acid and ethylene glycol monomers that constitute PET. It is estimated that from 375 to 500 million pounds of polyethylene terephthalate were used for beverage bottles in 1980, for example. More recently, more than 2.4 billion pounds of plastic bottles were recycled in 2008. Although the amount of plastic bottles recycled in the U.S. has grown every year since 1990, the actual recycling rate remains steady at around 27 percent. Recent legislation in several states requiring a deposit refundable upon return of all empty beverage containers has established an ongoing procedure for collecting and separating polyethylene terephthalate containers which must be recycled or otherwise disposed of in an economical manner. Additionally, many municipalities have implemented voluntary or mandatory recycling programs in conjunction with trash pickup and disposal.

PET beverage containers cannot be reused since the elevated temperatures required for sterilization deforms the container. PET containers can, however, be ground into small pieces for use as a filler material or remelted for formation of different articles. Such recycled material may be referred to interchangeably herein as “recycled PET”, “scrap PET”, “waste PET”, and/or “rPET”. The polyethylene terephthalate recovered by such processes contains impurities, such as pigment, paper, other undesirable polymers and metal from caps. Consequently, applications for polyethylene terephthalate reclamation by mechanical means are limited to non-food uses and low purity molded products.

In the past, several different techniques have been proposed for recovering pure or isolated terephthalic acid and ethylene glycol monomers from waste polyethylene terephthalate. One known technique involves, for example, the depolymerization of polyethylene terephthalate by saponification. Saponification is the hydrolysis of an ester under basic conditions to form an alcohol and the salt of a carboxylic acid (carboxylates).

In one known approach for saponification, polyethylene terephthalate is reacted with an aliphatic alcohol and a dialkyl terephthalate is recovered. This approach is exemplified in U.S. Pat. Nos. 3,321,510, 3,403,115 and 3,501,420, all of which are hereby incorporated by reference in their entirety. In a second known approach, polyethylene terephthalate is reacted with an aqueous solution of an alkali metal hydroxide or carbonate (usually sodium hydroxide) at an elevated temperature to yield a water soluble salt of terephthalic acid and ethylene glycol. The reaction product is acidified to liberate terephthalic acid which is water insoluble and the terephthalic acid precipitate is separated by filtration or the like. This approach is exemplified by U.S. Pat. Nos. 3,377,519, 3,801,273 and 3,956,088, all of which are hereby incorporated by reference in their entirety. U.S. Pat. No. 3,544,622, the entire contents of which is hereby incorporated by reference in its entirety, similarly discloses a variation to previously known approaches wherein the reaction is carried out under conditions to produce a water insoluble salt of terephthalic acid which is separated, washed and then acidified to produce terephthalic acid. Additional patents have also been issued on various improvements to these processes, such as U.S. Pat. Nos. 5,414,107, 5,223,544, 5,328,982, 5,045,122, 5,710,315, 5,532,404, 6,649,792, 6,723,873, 6,255,547, 6,580,005, 6,075,163, 7,173,150, 6,770,680, 7,098,299, and 7,338,981, the entire contents of each of which are hereby incorporated by reference in their entirety.

Empty beverage containers obtained from consumers may have aluminum caps lined with polyvinyl chloride or the like, wrap around polypropylene coated paper labels bonded to the surface with a polyvinyl acetate adhesive, residual sugars and, in some cases, polyethylene base caps for strengthening purposes. Without costly controls, reaction conditions in the saponification processes disclosed in the above-noted patents tend to cause some dissolution of these extraneous materials which then become impurities in the recovered terephthalic acid and require costly purification. Therefore, various approaches have been considered for removing these materials from the containers prior to grinding or separating them from the polyethylene terephthalate after grinding. Such separation procedures represent a significant increase in the overall cost of recovery as well as an energy inefficient means of recycling the waste PET. Thus, while such saponification methodologies for the recycling of PET into its monomer constituents are generally considered to be successful, it is an expensive and economically inefficient way in which to obtain such monomers for producing new PET polymers for use.

### SUMMARY OF THE INVENTION

The presently claimed and disclosed inventive concept(s) provide a functionalized oligomeric form of polyethylene terephthalate. In particular, the functional group is selected from the group consisting of a hydroxyl group, an amino group, carbonyl group and combinations thereof. The functionalized oligomeric form of polyethylene terephthalate can be made from a simple and efficient process for recovering oligomeric raw materials from polyester waste products in economical yields and high purity form. In one embodiment, the process is a saponification process. In a second embodiment, the process is a glycolysis process for recovering polyethylene terephthalate oligomers in economical yields from used polyethylene terephthalate beverage containers. The high purity terephthalic acid oligomers can dissolve in various organic solvents and thereafter provide a functionalized backbone for the production of polymeric based products. An example of the use of such polyethylene terephthalate oligomers would produce polyurethane dispersions (PUDs) and polyurethane resins (PURs),

### BRIEF SUMMARY OF THE DRAWINGS

FIG. 1 is a modulated differential scanning calorimetry (“mDSC”) analysis of the white precipitate from the first reaction. The derivative of the heat capacitance (J/(g° C.²)) indicated the presence of a melt at 120.95° C., indicating the presence of a small amount of oligomer.

FIG. 2 is liquid chromatography-mass spectrometry analysis of fully digested rPET.

FIG. 3 details liquid chromatography-mass spectrometry analysis of digested rPET having a MW distribution of 277-656 daltons.

FIG. 4 is a Fourier Transform Infrared Spectroscopy (FTIR)/Hyperion Reflectance analysis of the white precipitate from the first reaction.

FIG. 5 is an mDSC analysis of the white precipitate from the second reaction. The derivative of the non-reversible capacitance shows copious amounts of reactivity.

FIG. 6 is a Fourier Transform Infrared Spectroscopy (FTIR)/Hyperion Reflectance library match of FIG. 4. The top spectrum is of the white precipitate shown in FIG. 4, the matching bottom spectrum is Amoco TA-12, Terephthalic Acid.

FIG. 7 is an mDSC analysis of the white precipitate from the second reaction. The linear derivative of the non-reversible heat capacitance and reversible heat flow indicates that the white precipitate has reacted completely.

FIG. 8 shows the GPC data that illustrates that the rPET has been digested from a MW of 41,253 to a MW of 338-369 daltons.

FIG. 9 is a graphical representation of the effect on reaction time by the addition of water.

FIG. 10 is a graphical representation of the effect on reaction time by varying the amount of catalyst (i.e., zinc acetate) used in the reaction.

FIG. 11 is a graphical representation of the effect on reaction time versus the amount of catalyst.

FIG. 12 is a graphical representation of the effect of predigested rPET (Sample Ref. No. 188-17) on the digestion of green rPET.

FIG. 13 is a graphical representation of the effect of predigested rPET on the digestion of green rPET.

FIG. 14 is a graphical representation of the effect of predigested rPET (Sample Ref. No. 733-12, MW=1004) in a rPET digestion reaction.

FIG. 15 is a graphical representation of the effect of predigested rPET (Sample Ref. No. 188-17, MW=3381) in a rPET digestion reaction.

FIG. 16 is a graphical representation of the effect of predigested rPET on a clear rPET digestion.

FIG. 17 is a graphical representation of ¹H NMR spectra of an isolated white solid obtained in accordance with the present disclosure.

FIG. 18 is a graphical representation of ¹H NMR spectra over a 8.0-8.2 ppm range of an isolated white solid obtained in accordance with the present disclosure.

FIG. 19 is a graphical representation of ¹H NMR spectra over a 4.2-5.1 ppm range of an isolated white solid obtained in accordance with the present disclosure.

FIG. 20 is a graphical representation of ¹H NMR spectra over a 3.2-3.8 ppm range of an isolated white solid obtained in accordance with the present disclosure.

FIG. 21 is a graphical representation of ¹³C NMR spectra over a 0-220 ppm range of an isolated white solid obtained in accordance with the present disclosure.

FIG. 22 is a graphical representation of ¹³C NMR spectra over a 128-160 ppm range of an isolated white solid obtained in accordance with the present disclosure.

FIG. 23 is a graphical representation of ¹³C NMR spectra over a 58-68 ppm range of an isolated white solid obtained in accordance with the present disclosure.

FIG. 24 is a graphical representation of ¹H NMR spectrum of an isolated white solid plus 2 drops of ethylene glycol obtained in accordance with the present disclosure.

FIG. 25 is a graphical representation of ¹H NMR spectrum of an isolated white solid plus 2 drops of ethylene glycol obtained in accordance with the present disclosure.

FIG. 26 is a graphical representation of ¹H NMR spectrum of an isolated white solid plus 2 drops of ethylene glycol obtained in accordance with the present disclosure.

FIG. 27 is a graphical representation of ¹H NMR spectrum of ethylene glycol in DMSO-d6.

FIG. 28 is a graphical representation of ¹H NMR spectrum of ethylene glycol in DMSO-d6.

FIG. 29 is a graphical representation of ¹H NMR spectrum of DMSO-d6 Blank.

FIG. 30 is a graphical representation of ¹H NMR spectrum of DMSO-d6 Blank.

FIG. 31 is a graphical representation of ¹H NMR spectrum over a 0-12 ppm range of DMSO-d6 Blank.

FIG. 32 is a graphical representation of ¹H NMR spectra over a 6-12 ppm range of a green semi-solid isolated from green toluene filtrate in accordance with the present disclosure.

FIG. 33 is a graphical representation of ¹H NMR spectra over a 6.8-8.8 ppm range of a green semi-solid isolated from green toluene filtrate in accordance with the present disclosure.

FIG. 34 is a graphical representation of ¹HNMR spectra over a 4.1-5.4 ppm range of a green semi-solid isolated from green toluene filtrate in accordance with the present disclosure.

FIG. 35 is a graphical representation of ¹H NMR spectra over a 2.8-4.0 ppm range of a green semi-solid isolated from green toluene filtrate in accordance with the present disclosure.

FIG. 36 is graphical representation of ¹H NMR spectra over a 1.8-2.7 ppm range of a green semi-solid isolated from green toluene filtrate in accordance with the present disclosure.

FIG. 37 is a graphical representation of ¹H NMR spectra over a 1.25-1.95 ppm range of a green semi-solid isolated from green toluene filtrate in accordance with the present disclosure.

FIG. 38 is a graphical representation of ¹H NMR spectra over a 0-1 ppm range of a green semi-solid isolated from green toluene filtrate in accordance with the present disclosure.

FIG. 39 is a graphical representation of ¹H NMR spectra over a 0-1.7 ppm range of a green semi-solid isolated from green toluene filtrate in accordance with the present disclosure.

FIG. 40 is a graphical representation of ¹³C NMR spectra over a 0-220 ppm range of a green semi-solid isolated from green toluene filtrate in accordance with the present disclosure.

FIG. 41 is a graphical representation of ¹³C NMR spectra over a 140-220 ppm range of a green semi-solid isolated from green toluene filtrate in accordance with the present disclosure.

FIG. 42 is a graphical representation of ¹³C NMR spectra over a 80-140 ppm range of a green semi-solid isolated from green toluene filtrate in accordance with the present disclosure.

FIG. 43 is a graphical representation of ¹³C NMR spectra over a 112-140 ppm range of a green semi-solid isolated from green toluene filtrate in accordance with the present disclosure.

FIG. 44 is a graphical representation of ¹³C NMR spectra over a 10-75 ppm range of a green semi-solid isolated from green toluene filtrate in accordance with the present disclosure.

FIG. 45 is a graphical representation of ¹³C NMR spectra over a 56-75 ppm range of a green semi-solid isolated from green toluene filtrate in accordance with the present disclosure.

FIG. 46 is a graphical representation of ¹³C NMR spectra over a 13-34 ppm range of a green semi-solid isolated from green toluene filtrate in accordance with the present disclosure.

FIG. 47 is a graphical representation of ¹³C NMR spectra over a 28-30 ppm range of a green semi-solid isolated from green toluene filtrate in accordance with the present disclosure.

### DETAILED DESCRIPTION

Before explaining at least one embodiment of the presently disclosed and claimed inventive concept(s) in detail, it is to be understood that the presently disclosed and claimed inventive concept(s) is not limited in its application to the details of construction, experiments, exemplary data, and/or the arrangement of the components set forth in the following description or illustrated in the drawings. The presently disclosed and claimed inventive concept(s) is capable of other embodiments or of being practiced or carried out in various ways. Also, it is to be understood that the phraseology and terminology employed herein is for purpose of description and should not be regarded as limiting.

Disclosed herein are functionalized polyethylene terephthalate (“PET”) polymers, functionalized derivatives of PET (“fPET”), and methods for digesting recycled (“rPET”) into lower molecular weight functionalized digested PET materials (“dfPET”). Methods of manufacturing and making these dfPET polymers are disclosed and claimed in U.S. Provisional Patent Application 61/337,568, filed on Feb. 9, 2010 and U.S. Provisional Application Ser. No. 61/404,621, filed Oct. 6, 2010, the entirety of both of which are hereby expressly incorporated herein by reference.

Table A discloses general chemical structures representing the presently disclosed inventive dfPET. n can be any positive integer greater than 1—for example n is a positive integer from 2 to 500,000. R¹ and R² are independently hydrocarbons having at least one functional group selected from the group consisting of a hydroxyl group, an amino group, a carbonyl group and combinations thereof. For example, but not by way of limitation, R¹ and R² may be independently selected from the group consisting of H, OH, NH₂, C═O, hydroxyl terminated hydrocarbons, diol terminated hydrocarbons, amine terminated hydrocarbons, diamine terminated hydrocarbons, carbonyl terminated hydrocarbons, amino alcohols consisting of a hydrocarbon terminated with an amine group and an alcohol group, hydroxylcarboxylic acids consisting of a hydrocarbon terminated with a hydroxyl group and a carboxyl group, and amides consisting of a hydrocarbon terminated with a carbonyl group and an amine group.

In addition to the functional groups described above, R¹ and R² can be independently hydrocarbons containing other functional groups such as a halo group, a thiol group, a phosphate group, and an ether group. The hydrocarbons can be alkanes, alkenes, cycloalkanes, cycloalkenes and aromatics.

The hydroxyl terminated hydrocarbons includes, for example but not by way of limitation, hydroxyl terminated alkanes and branched hydroxyl terminated alkanes. The diol terminated hydrocarbons includes, for example but not by way of limitation, diol terminated alkanes and branched diol terminated alkanes. The amine terminated hydrocarbons includes, for example but not by way of limitation, amine terminated alkanes branched amine terminated alkanes. The diamine terminated hydrocarbons includes, for example but not by way of limitation, diamine terminated alkanes branched diamine terminated alkanes. The amino alcohols include, for example but not by way of limitation, alkanes terminated with an amine group and an alcohol group and branched amino alcohols terminated with an amine group and an alcohol group. The hydroxyl carboxylic acids include, for example but not by way of limitation, alkanes terminated with a carboxyl group and an alcohol group and branched hydroxyl carboxylic acids terminated with a carboxyl group and an alcohol group. The amides include, for example but not by way of limitation, alkanes terminated with an amine group and a carbonyl group and branched amides terminated with an amine group and a carbonyl group.

In one embodiment, R¹ and R² can be independently a linear or branched C₂-C₁₈ alkane having at least one functional group selected from the group consisting of a hydroxyl group, an amino group, a carbonyl group and combinations thereof. In another embodiment, R¹ and R² can be independently a linear or branched C₂-C₈ alkane having at least one functional group selected from the group consisting of a hydroxyl group, an amino group, a carbonyl group and combinations thereof.

TABLE A

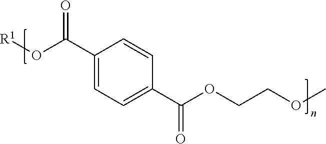

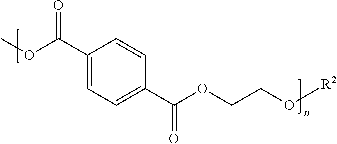

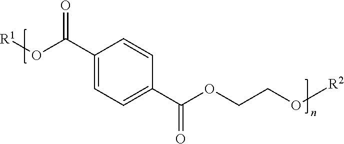

The oligomeric form of polyethylene terephthalate can be produced from a reaction of polyethylene terephthalate, a glycolysis agent and a catalyst. In one embodiment, the glycolysis agent can be a polyol. The polyols may be, for example but by way of limitation, ethylene glycol, diethylene glycol, triethylene glycol, propylene glycol, 1,3-propanediol, 2-methyl-1,3-propanediol, 1,3-butanediol, 1,4-butanediol, 1,5-pentanediol, 1,6-hexanediol, 2,2-diethyl-1,3-propanediol, 1,7-heptanediol, 1,8-ocatanediol, 1,9-nonanediol, 1,4-cyclohexanedimethanol, 2-butyl-2-ethyl-1,3-propanediol, 2-butyl-2-diethyl-1,3-propanediol, 3-methyl-1,5-pentanediol, 2,2,4-trimethylpentanediol, 1,4-dimethyolcyclohexane, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol, neopentyl glycol, cyclohexanediol, hydrogenated bisphenol A, glycerol, trimethylolpropane, trimethylolethane, diglycerine, triglycerine, pentaerythritol, dipentaerythritol, sorbitol, 1,4-polyisoprenediol, 1,2-polybutadinediol, polybutanediol.

The oligomeric form of polyethylene terephthalate can comprise a reaction product of polyethylene terephthalate, a glycolysis agent, an amine and a catalyst. In one embodiment, the amine can be hexamethyltetraamine. In another embodiment, the amine can be a diamine. The diamine can be an aliphatic diamine, an aromatic diamine and an alicyclic diamine. The aliphatic diamines may be, for example but not by way of limitation, ethylene diamine, trimethylene diamine, 1,2-diaminopropane, 1,3-diaminopropane, tetramethylene diamine, pentamethylene diamine, hexamethylene diamine, 1,8-diaminooctane, dodecamethylene diamine, and 2,2,4-trimethyl hexamethylene diamine.

The aromatic diamines may be, for example but not by way of limitation, p-phenylene diamine, o-phenylene diamine, m-phenylene diamine, m-toluoylene diamine, p-xylene diamine, m-xylene diamine, 4,4′-diamino biphenyl, 3,3′-dimethyl-4,4′-diamino biphenyl, 3,3′-dichloro-4,4′-diamino biphenyl, 4,4′-diamino diphenyl ether, 3,4′-diamino diphenyl ether, 4,4′-diamino diphenyl propane, 4,4′-diamino diphenyl sulfone, 4,4′-diamino diphenyl sulfide, 4,4′-diamino benzanilide, 3,3′-dimethyl-4,4′-diamino diphenyl methane, 3,3′-diethyl-4,4′-diamino diphenyl methane, 4,4′-diamino anthraquinone, 3,3′-dimethoxybenzidine, α,α′-bis(4-aminophenyl)-p-isopropylbenzene, 1,5-diamino naphthalene, and 2,6-diamino naphthalene.

The alicyclic diamines may be, for example but not by way of limitation, 1,3-diamino cyclohexane, 1,4-diamino cyclohexane, 1,3-bis(aminomethyl)cyclohexane, isophorone diamine, piperazine, 2,5-dimethyl piperazine, bis(4-aminocyclohexyl)methane, bis(4-aminocyclohexyl)propane, 4,4′-diamino-3,3′-dimethyl dicyclohexylmethane, α,α′-bis(4-aminocyclohexyl)-p-diisopropylbenzene, α,α′-bis(4-aminocyclohexyl)-m-diisopropylbenzene, and methane diamine. Any one of, or any combination of, the diamine compounds as described above may be used. The glycolysis agents can be the same as those described previously.

The functionalized oligomeric form of polyethylene terephthalate can also be obtained from a reaction of polyethylene terephthalate, a glycolysis agent, a diacid and a catalyst. In one embodiment, the diacid is selected from the group consisting of oxalic acid, malic acid, malonic acid, tartaric acid, glutaric acid, succinic acid, fumaric acid, adipic acid, sebacic acid, maleic acid, azelaic acid, isophthalic acid, terephthalic acid, phthalic acid, terephthalic acid dichloride, 1,4-cyclohexanedicarboxylic acid, 1,3-cyclohexanedicarboxylic acid, naphthalenedicarboxylic acid, 4,4′-biphenyldicarboxylic acid, diphenylmethane-4,4′-dicarboxylic acid, and combinations thereof.

In addition to diacids, a compound having both a hydroxyl group (s) and a carboxylic group (s) can also used in the reaction or can be combined with diacids in the reaction. The hydroxycarboxylic acids may be, for example but by way of limitation, glycolic acid, 3-hydroxylactic acid, 4-hydroxybutyric acid, 4-hydroxyvaleric acid, 6-hydroxycaproic acid, hydroxybenzoic acid, hydroxypivalic acid, 1,2-dihydroxystearic acid, 2,2-dimethylolpropinoic acid, 2,2-dimethylolbutanoic acid, 2,2-dimethylolpentanoic acid, 2,2-dimethylolhexanoic acid, and 2,2-dimethyloloctanoic acid. The glycolysis agents can be the same as those described previously.

Similarly, the functionalized oligomeric form of polyethylene terephthalate can be produced from a reaction of polyethylene terephthalate, a glycolysis agent, an anhydride and a catalyst. In one embodiment, the anhydride is selected from the group consisting of propionic anhydride, maleic anhydride, succinic anhydride, methacrylic anhydride, glutaric anhydride, trimelletic anhydride, pyromellitic anhydride, phthalic anhydride, tetrabromophthalic anhydride, tetrachlorophthalic anhydride, hexahydrophthalic anhydride, tetrahydrophthalic anhydride, and combinations thereof. The glycolysis agents are the same as those described previously.

The polyethylene terephthalate is selected from the group consisting of recycled polyethylene terephthalate, virgin polyethylene terephthalate and combinations thereof. The catalyst can be metal acetates. In one embodiment, the catalyst is zinc acetate.

Table B shows the resulting dfPET polymeric structures derived from several glycolysis agents and propionic anhydride, in which n can be any positive integer greater than 1—for example n is a positive integer from 2 to 500,000.

TABLE B

| Glycolysis Agent | Resulting dfPET Structure |
|---|---|
| Neopentyl glycol | 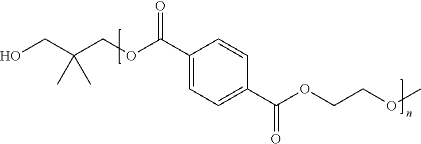 |
| Ethylene Glycol | 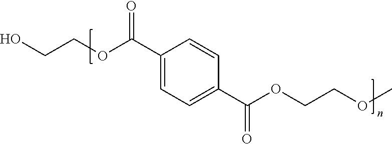 |
| Glycerol | 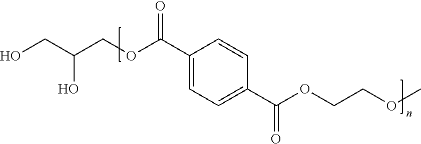 |
| Propionic Anhydride | 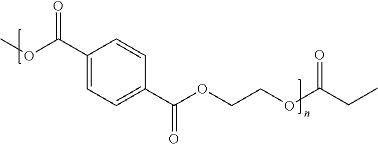 |

The oligomeric form of polyethylene terephthalate containing hydroxyl group(s) is a very important intermediate and can be further converted to other widely useful chemical products. In one particular aspect, the oligomeric form of polyethylene terephthalate containing hydroxyl group(s) can be converted to the corresponding alkenes by dehydration in the presence of a catalyst composition. The catalyst composition can be an acid catalyst. In one embodiment, the catalyst composition is concentrated sulfuric acid or concentrated phosphoric acid. The catalyst composition can also be a solid acid catalyst. The solid acid catalyst composition for dehydrating an alcohol was substantially as described in U.S. Patent Pub. 2011/0098519, the entire disclosure of which is hereby incorporated by reference. The solid acid catalyst may be, for example, and without limitation, a bulk oxide. In one embodiment, the bulk oxide may be, for example, and without limitation, alumina, zirconia, titania, silica or niobia.

The solid acid catalyst may be, for example, and without limitation, a zeolite. The meaning of the expression “zeolite” would be understood to those of ordinary skill in the art. A zeolite may include, for example, and without limitation, a hydrated aluminosilicate of the alkaline and alkaline earth metals. Suitable zeolites would be understood to and can be determined by those of ordinary skill in the art. In an embodiment, the zeolite may be, for example and without limitation, of natural or synthetic origin. In an embodiment, the zeolite may be, for example and without limitation, crystalline. In an embodiment, the zeolite may be, for example, and without limitation, a pentasil-type zeolite. In an embodiment, the zeolite may be, for example and without limitation, HY, H-BETA, H-Mordenite or ZSM-5 zeolite. The expressions “HY”, “H-BETA”, “H-Mordenite” and “ZSM-5” would be understood to those of ordinary skill in the art. In an embodiment, the zeolite may be, for example and without limitation, ZSM-5 zeolite. The expression “ZSM-5” is used interchangeably with the expression “H-ZSM-5” throughout this entire specification. A modifying agent can be added into the above solid acid catalyst to enhance the surface acidity. Examples of the modifying agent include, but are not limited to, phosphate or sulfate compounds such as phosphoric acid or sulfuric acid, or a derivative thereof, or a transition metal oxide such as tungsten trioxide, ZrO₂ and MoO₃, or a derivative thereof,

The resulted alkenes can be used as chemical intermediates or building blocks to produce other useful products applied in a number of industries. Since the alkenes can be produced from waste products, such as beverage containers, made from polyethylene terephthalate, the production of valued chemicals from the alkenes is attracting considerable interest. In this regard, production of alkene from oligomeric form of polyethylene terephthalate containing hydroxyl group(s) is a promising approach.

Metal alkoxides are widely used in industry as catalysts and stoichiometric reagents. These reagents are used in diverse reaction chemistries such as alkylation, isomerization, rearrangements, condensations, transesterifications and eliminations. The oligomeric form of polyethylene terephthalate containing hydroxyl group(s) can react with a metal reagent or a metal salt to form the corresponding metal alkoxide. In one particular aspect, the oligomeric form of polyethylene terephthalate containing hydroxyl group(s) reacts with at least a stoichiometric amount of a metal reagent. The metal reagent can be, but are not limited to a Group I metal, a Group II metal, zinc, a metal alloy of a Group I metal, a metal alloy of a Group II metal, a compound of zinc, or combinations thereof. In one embodiment, a metal reagent used includes K, Li, Na, Cs, Mg, Ca or Zn. In the case that a metal reagent is used, the reaction takes place above the melting point of the metal. The synthesis and isolation of metal alkoxides using a metal reagent was substantially as described in U.S. Pat. No. 6,444,862, the entire disclosure of which is hereby incorporated by reference.

The metal alkoxide can also be formed by reaction of the oligomeric form of polyethylene terephthalate containing hydroxyl group(s) with a metal salt. In one embodiment, the metal salt is a metal halide. Typically, a metal halide reacts with the oligomeric form of polyethylene terephthalate containing hydroxyl group(s) in the presence of ammonia. Ammonia is used to remove the halide. Optionally, inert solvents such as benzene, toluene, xylene, octane, or cyclohexane may be used as solvent or cosolvent. The ammonia reactant is sparged into the reaction medium until substantially all of the ammonium halide is formed.

Ethers are commercially important compounds and widely used with respect to solvents, propellants, fillers, food additives, fuel additives, cleaners, health care formations and manufacture of polymers, etc. Ethers can also be found in many familiar commercial products from hair spray to cosmetics. In the presently disclosed and claimed inventive concept(s), the oligomeric form of polyethylene terephthalate containing hydroxyl group(s) can be converted to the corresponding symmetric ether by condensation in the presence of an acid catalyst. In one embodiment, a strong acid (such as sulfuric acid) is added to the solution of the oligomeric form of polyethylene terephthalate containing hydroxyl group(s) and then the reaction mixture is heated.

The symmetric ether can also be produced from the oligomeric form of polyethylene terephthalate containing hydroxyl group(s) using metal oxides as catalyst. First, a feedstock with the oligomeric form of polyethylene terephthalate containing hydroxyl group(s) is heated to a temperature greater than about 150 degree Celsius. And then the feedstock is passed through a catalyst comprising a metal oxide. The metal oxides can be, but are not limited to, zirconia, hafnia, titania, alumina, or the combinations thereof. In some embodiments, the metal oxide is selected from the group consisting of titania and alumina. The ether synthesis using a metal oxide as catalyst was substantially as described in U.S. Patent Pub. 2008/0319236, the entire disclosure of which is hereby incorporated by reference.

The oligomeric form of polyethylene terephthalate containing hydroxyl group(s) can also be converted to an unsymmetrical ether using the metal alkoxide of the oligomeric form of polyethylene terephthalate produced previously through Williamson ether synthesis. This synthesis involves converting an alkoxide ion into an ether by reaction with a hydrocarbyl halide. Alternatively, the ether can be produced by reaction of the oligomeric form of polyethylene terephthalate containing hydroxyl group(s) with a hydrocarbyl halide in the presence of a substantial, stoichiometric excess of water soluble, hygroscopic base. In one embodiment, the water soluble, hydroscopic base is sodium hydroxide. First, the oligomeric form of polyethylene terephthalate containing hydroxyl group(s) reacts with at least a 20 molar percent stoichiometric excess of a water-soluble hygroscopic base to form an alkoxide anion. The alkoxide anion is then reacted with a source of an alkyl moiety such as a hydrocarbyl halide or the like to form ether. The ether synthesis from alkoxide anions was substantially as described in U.S. Patent Pub. 2010/0280277, the entire disclosure of which is hereby incorporated by reference.

The oligomeric form of polyethylene terephthalate containing hydroxyl group(s) can react with organic silyl halides in the presence of acid acceptor to produce the corresponding silyl ether. Silyl ethers are usually used as protecting groups for alcohols in organic synthesis, especially for synthesis of pharmaceutical ingredients.

Esters encompass a large family of organic compounds with broad applications in medicine, biology, chemistry and industry. Esters are produced by reaction of acids with compounds containing hydroxyl group(s). In the present presently disclosed and claimed inventive concept(s), the oligomeric form of polyethylene terephthalate containing hydroxyl group(s) can react with various inorganic and organic acids to form the corresponding esters. For example, the oligomeric form of polyethylene terephthalate containing hydroxyl group(s) can react with inorganic acids such as nitric acid, phosphoric acid and sulfuric acid to form the corresponding nitrate, phosphate and sulfate, respectively. The oligomeric form of polyethylene terephthalate containing hydroxyl group(s) can also react with organic acids and anhydrides to form corresponding organic esters in the presence of an inorganic acid catalyst. The acids include organic monoacids and diacids. The esters can be converted to a thionoester using Lawesson's reagent.

The oligomeric form of polyethylene terephthalate containing hydroxyl group(s) may be converted to a sulfonate by reacting the oligomeric form of polyethylene terephthalate containing hydroxyl group(s) with an appropriate sulfonic acid. The sulfonic acid can be an alkyl sulfonic acid, an aryl sulfonic acid, an alkyl aryl sulfonic acid or combinations thereof.

The oligomeric form of polyethylene terephthalate containing hydroxyl group(s) can react with sulfonyl halides to form the corresponding sulfonates. The sulfonyl halides can be tosyl chloride, brosyl, mesyl and trifyl. As a result, a tosylate, a brosylate and a triflate can be produced. These are important chemical intermediates used widely in organic synthesis. For example, tosylate and triflate can be converted to the corresponding amines and esters.

The oligomeric form of polyethylene terephthalate containing hydroxyl group(s) can be also converted to the corresponding halides. In one embodiment, the oligomeric form of polyethylene terephthalate containing hydroxyl group(s) can react with a hydrogen halide to form the corresponding halide in the presence of sulfuric acid. The reaction is carried out with stoichiometric excess of the hydrogen halide relative to the oligomeric form of polyethylene terephthalate containing hydroxyl group(s).

In another embodiment, the oligomeric form of polyethylene terephthalate containing hydroxyl group(s) can react with thionyl halide in the presence of base catalyst to generate the corresponding halide. The oligomeric form of polyethylene terephthalate containing hydroxyl group(s) is dissolved in aprotic polar solvent in the presence of base catalyst then is slowly added thionyl halide at low temperature ranged from about −20° C. to about 10° C. The halide is selected from F, Cl, Br or I. In one embodiment, Cl or Br is used. Among the thionyl halide, thionyl chloride and thionyl bromide are commercially available. Thionyl chloride is recommended due to easy purchase in large scale and less heat generation during the reaction. The aprotic polar solvents used include, but are not limited to, acetonitrile, methylene chloride, chloroform, carbon tetrachloride and diethyl ether. Among them, acetonitrile, methylene chloride or chloroform is more desirable.

Either organic or inorganic salts can be used as the base catalyst even in excess amount. Examples of the organic base include, but are not limited to, triethylamine, tripropylamine, N,N-diisopropylamine, and pyridine. Examples of the inorganic base include, but are not limited to, potassium hydroxide, sodium carbonate and potassium carbonate. In yet another embodiment, the oligomeric form of polyethylene terephthalate containing hydroxyl group(s) can react with phosphorous halides to form the corresponding halides.

The halides are very important chemical intermediates that can be used to generate other useful chemicals. For example, the halides can be used to make the corresponding amine derivatives, which are widely used as intermediates for the synthesis of various organic compounds as well as pharmaceutical and agro-chemical compounds. In one embodiment, the halide is dissolved in aprotic polar solvent and reacts with amine in the presence of base catalyst. The reaction can be carried out at the temperature ranged from about 0° C. to about 200° C., being recommended to reflux under the pressure ranged from about 1 to about 100 atm depending on the amine. The polar solvents include, but are not limited to, acetonitrile, toluene, dimethylformamide, dimethylacetamide, dioxane, tetrahydrofuran, and pyridine. Among them, acetonitrile and dimethylacetamide are desirable. The basic catalysts include either organic base such as pyridine, triethylamine, diisopropylamine or the inorganic base such as sodium carbonate, potassium carbonate, calcium carbonate, sodium hydroxide, potassium hydroxide, sodium hydride, potassium hydride, calcium hydride, sodium methoxide, and sodium ethoxide. Among them, sodium carbonate and potassium carbonate are recommended. Any amine compound can be used for the reaction. In one embodiment, alkyl amine and cycloalkyl amine are used. The halides converting to amines was substantially as described in U.S. Pat. No. 6,566,525, the entire disclosure of which is hereby incorporated by reference.

The oligomeric form of polyethylene terephthalate containing hydroxyl group(s) can also be oxidized to form the corresponding aldehydes, ketones and acids using oxidized agents. The oxidized agents can be oxygen (air) or hydrogen peroxide. In one specific aspect, the oligomeric form of polyethylene terephthalate containing hydroxyl group(s) can be oxidized by oxygen or air in the presence of catalysts. The oxidation can be carried out using ruthenium, cobalt, copper, palladium, and platinum metal catalysts with additives such as potassium carbonate, sodium bicarbonate, pyridine, molecular sieves and phenanthroline. Stoichiometric metal oxidants such as chromium (VI) compound and active manganese dioxide have also been widely used as oxidation catalysts. In one embodiment, a ruthenium-carrying alumina can be used a catalyst. In another embodiment, a ruthenium compound and a dioxybenzene or its oxidant is used as a catalyst. In yet another embodiment, a manganese-containing octahedral molecular sieve can be used as a catalyst. All these catalysts and the oxidation processes were substantially as described in U.S. Pat. Nos. 7,169,954; 6,486,357; and 6,166,264, the entire disclosures of which are hereby incorporated by references.

The oligomeric form of polyethylene terephthalate containing hydroxyl group(s) can be oxidized by hydrogen peroxide in the presence of a catalyst. In one embodiment, the catalysts can be, but are not limited to tungsten catalyst such as peroxotungstate, sodium tungstate, and tungstic acid. The oxidation of alcohol with use of hydrogen peroxide and tungsten catalyst was substantially as described in U.S. Patent Pub. 2008/0269509, the entire disclosure of which is hereby incorporated by reference. In another embodiment, the catalyst is rhenium based catalyst with a co-catalyst selected from the group consisting of HBF₄ and salts thereof. The rhenium based catalyst can be an unsupported and a supported rhenium based catalyst. The supported rhenium based catalysts usually comprise an inert polymeric matrix (support) and a rhenium compound (active part of the catalyst). Examples of rhenium compounds include, but are not limited to, ReO₃, Re₂O₇, CH₃ReO₃, a C₂ to C₂₀ alkyl rhenium oxide, a C₃ to C₁₀ cycloalkyl rhenium oxide. The oxidation of alcohol with use of hydrogen peroxide and rhenium based catalyst was substantially as described in U.S. Patent Pub. 2011/0124889, the entire disclosure of which is hereby incorporated by reference. The produced aldehydes or ketones can be further converted to the corresponding alkenes by reaction with a triphenyl phosphonium ylide (often called a Wittig reagent).

The oligomeric form of polyethylene terephthalate containing hydroxyl group(s) can react with hydrogen sulfide in the presence of a catalyst to produce the corresponding thiol. In one embodiment, the catalyst comprises a support, a base and a metal compound. The support is a catalytically active carrier that contains base and/or acid active sites. Examples of the supports include, but are not limited to, alumina, zirconia, silica, titania, aluminum-silicate (zeolites) and magnesia-aluminates. The base is an alkali metal, alkaline earth metal bicarbonate, carbonate, oxide, or hydroxide. In one embodiment, alkali metal bases and hydroxides are used. Examples of suitable bases include, but are not limited to, sodium hydroxide, potassium hydroxide, magnesium hydroxide, calcium hydroxide, sodium bicarbonate, sodium carbonate, magnesium oxide and calcium oxide. In one embodiment, potassium hydroxide and rubidium hydroxide are used as base.

The metal compound is an acid or an alkali metal or alkaline earth metal salt thereof. The metals are Group III to XII in the Periodic Table and include tungsten, molybdenum, chromium, manganese, titanium, zirconium, cobalt and nickel. In one embodiment, a tungsten compound is used. Examples of the metal compounds include, but are not limited to, WO₃, K₂WO₄, Na₂WO₄, MoO₃, K₂MoO₄, Na₂MoO₄, phosphotungstate, phosphomolybdate and silicotungstate. In one embodiment, WO₃ or K₂WO₄ are used. The synthesis of thiols from alcohols was substantially as described in U.S. Pat. No. 5,874,630, the entire disclosure of which is hereby incorporated by reference.

The thiol can be further converted to thioester condensate by reacting with acids in the presence of a solvent using a tetravalent hafnium compound as a condensation catalyst. Generally, the acids can be carboxylic acids including monocarboxylic acids, dicarboxylic acids, tricarboxylic acids, and tetracarboxylic acids. The tetravalent hafnium compound is a hafnium chloride (IV), a hafnium chloride (IV)(THF)₂, or hafnium (IV)t-butoxide. A solvent can be a polar solvent, a nonpolar solvent, or a combination of a polar and a nonpolar solvent. The nonpolar solvent is recommended. Examples of the nonpolar solvents include, but are not limited to, toluene, xylene, mesitylene, pentamethylbenzene, m-terphenyl, benzene, ethylbenzene, 1,3,5-tri-isoporpyl benzene, o-dichlorobenzene, 1,2,4-tricholobenzene, naphthalene, and 1,2,3,4-tetrahydronaphthalene (tetralin). The production of condensation thioester was substantially as described in U.S. Pat. No. 7,301,045, the entire disclosure of which is hereby incorporated by reference.

In addition, the oligomeric form of polyethylene terephthalate containing hydroxyl group(s) can be oxidized by oxygen to produce the corresponding hydroperoxide. The oligomeric form of polyethylene terephthalate containing hydroxyl group(s) can react with reducing agents to producing the corresponding alkanes and/or alkenes.

Broadly, the process of making these dfPET polymers presently claimed and disclosed inventive concept(s) includes the step of reacting polyethylene terephthalate scrap and/or waste, and/or virgin material and/or combinations thereof with ethylene glycol (i.e., a glycolysis agent) containing a catalyst at an elevated temperature and at atmospheric pressure for a sufficient time to decrease the molecular weight of the PET scrap to an oligomeric state. In one embodiment, the catalyst is a zinc acetate catalyst capable of decreasing the amount of activation energy for depolymerization of polyethylene terephthalate. In an additional step, precipitated PET oligomer is recovered from the reaction mixture and dried. In another embodiment, neopentyl glycol is used as the glycolysis agent. One of ordinary skill in the art would appreciate that other glycolysis agents such as, but not by way of limitation, glycerol and propionic anhydride, may also be used. As can be appreciated, the resulting dfPET polymeric material (and chemical structure) is determined by the glycolysis agent used and one of ordinary skill in the art, given the present disclosure, would appreciate and be capable of producing any specific dfPET material having a desired chemical structure.

While the process of the presently disclosed and claimed inventive concept(s) can be used to treat a wide variety of polyethylene terephthalate scrap or waste, it is particularly adaptable for processing used polyethylene terephthalate beverage containers. As used hereinafter, the term “scrap” means scrap, waste and/or used products of polyethylene terephthalate. The term “scrap” also includes within its definition varying sizes and shapes of waste and/or used products of polyethylene terephthalate. Scrap PET, as used herein, may include whole products made of PET (e.g., a beverage bottle) or further processed products made of PET. In one embodiment, the further processing includes the chipping or shredding of PET products in order to produce a scrap PET material suitable for use in the disclosed methodologies. Alternatively, the further processing may include nitrogen jet milling of the PET products in order to produce a scrap PET material having an average size of about 10 microns. One of ordinary skill in the art would appreciate that the further processing step may include a multitude of processing steps including, but not limited to, pin milling, jet milling, media milling, rolling and crushing, all of which would be understood to fall within the broad disclosure presented herein.

In one embodiment, further processing of the PET raw material is accomplished via milling. For example, recycled bulk PET (rPET) having a particle size in the range of 100-200 microns was obtained from Clean Tech Incorporated (Dundee, Mich.). This bulk recycled PET is formed from PET plastic bottles that are sorted by color, ground, washed and repelletized and dried under vacuum conditions. The recycled bulk PET was in the form of grayish pellets. Further processing for this embodiment entailed liquid nitrogen jet milling of the recycled bulk PET pellets according to the conditions outlined in Table 1 and performed by LiquaJet/The Jet Pulverizer Co. (Moorestown, N.J.). The processing steps performed by LiquaJet are proprietary methods kept as a trade secret by the company. Generally, the material was milled with liquid nitrogen in order to obtain a product having a desired state. The results of particle size shown in Table 1 were determined on a Wet Horiba Ri:1.5750 (HORIBA Ltd., Austin, Tex.).

TABLE 1

| | Avg. Particle Size of | |
|---|---|---|
| Test Condition | rPET in microns. | Grit |
| 1A | 64.4 | NO |
| 1B | 59.1 | NO |
| 2A | 33.1 | NO |
| 2B | 83.0 | NO |
| 3A (2A 2X) | 27.4 | NO |
| 4A | 51.6 | NO |
| 4B | 51.6 | YES |
| 5A | 108.5 | YES |
| 5B | 153.0 | YES |

After a second milling using the liquid nitrogen jet milling process, the rPET material was found to have an average size of 27.4 microns. Further processing (i.e., additional liquid jet milling steps) would achieve a specification of rPET material having an average size of less than about 33 microns and, more particularly, from about 7 to about 10 microns. Although such small sizes of rPET can be obtained, it was found that the process(es) according to the presently disclosed and claimed inventive concept(s) do not require such a small starting size of the rPET. Rather, it was found that rPET having a size of from about 25 microns to about 100 microns can be used and, more particularly, rPET scrap having a size of from about 50 microns to about 100 microns. Such sizes should not be considered as limiting, however, as the presently disclosed and claimed inventive concept(s) have been found to be suitably applied to rPET scrap having a size equal to or greater than 200 microns.

Chemical recycling of a plastic is by definition the recycling of a chemical by partially altering its chemical structure through a chemical reaction into a viable material. PET is produced by the reaction of ethylene glycol and terephthalic acid. The reaction between the acid and the glycol results in an ester linkage and water. Water is removed from the reaction kettle to continue polymerization thereby increasing the average molecular weight of the PET.

The depolymerization of the rPET into a reactive, lower melting point (mp) material was accomplished according to novel methodologies of the presently disclosed and claimed inventive concept(s). The molecular weight of the polymer is reduced until a molecular weight of 280-680 is achieved, for example. At this molecular weight the PET has the physical qualities of a lower melting point (mp) and increased reactivity useful for some applications.

An initial reaction scheme for depolymerization of the rPET into a reactive lower melting point material according to the presently disclosed and claimed inventive concept(s) utilized the reactant weights given in Table 2.

TABLE 2

| Reactants | Amount | |
|---|---|---|
| Deionized Water | 300 | ml |
| rPET (TABLE A - 1A) | 48.4393 | g |
| Anhydrous Sodium Acetate | 3.0263 | g |
| 50% by Weight Sodium Hydroxide | 30 | ml |

The reactants were all weighed and added to the reaction kettle. The kettle was set up with a stir bar and a condenser with cold water running through it. A heating mantle was used to heat the mixture as it was stirring. The temperature of the reaction kettle was maintained at a constant temperature of from about 150-175° C. The reaction was allowed to proceed for 6 hours at which time the rPET had completely dissolved. The reaction solution was then cooled to a uniform highly basic solution. A small sample of the cooled mixture was placed into a beaker and concentrated hydrochloric acid was added until a white precipitate formed.

More particularly, the procedure for the reaction of Table 2 was as follows:
1. 250 ml of water was added to the reaction kettle.
2. 48.4393 g of rPET (Table 1-Sample 1A) was added to the reaction kettle.
3. 3.0262 g of anhydrous sodium acetate catalyst was added to the reaction kettle.
4. 30 ml of 50% by weight sodium hydroxide was added.
5. The reaction was heated to a temperature of 150-175° C.
6. Water (350 mL) was added in two increments.
7. When the rPET was completely dissolved (after approximately 6 hours), the reaction kettle was allowed to cool to room temperature.
8. An aliquot of the solution was removed from the kettle and acidified with concentrated hydrochloric acid which afforded a white precipitate.

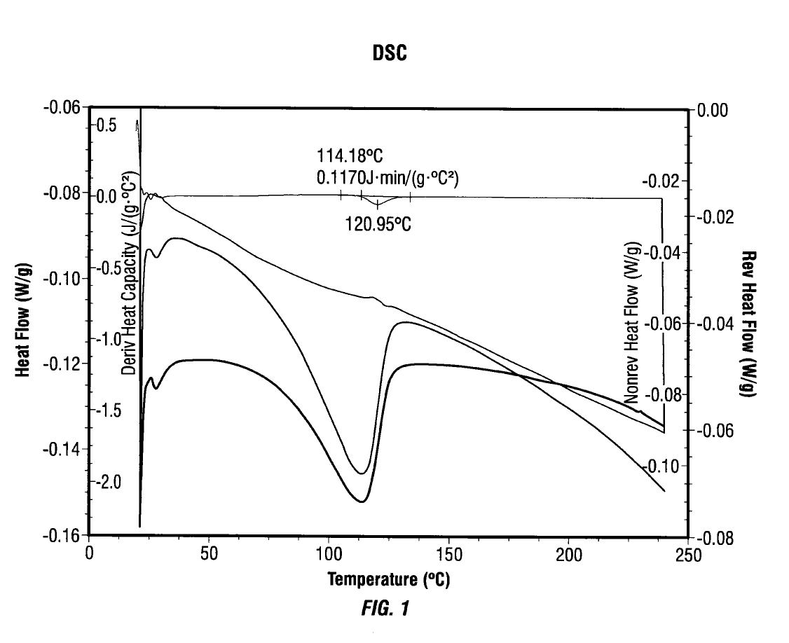

The white precipitate was dried and characterization by Differential Scanning calorimetry (DSC), FTIR, and LC-MS (Liquid Chromatography with Mass Spectrometric detection) revealed that the resultant products were primarily the monomers: ethylene glycol and terephthalic acid. Modulated DSC (mDSC) was used to determine the material's melting point. A small melt occurring at 109° C. indicated, moreover, that a minor amount of bis(2-hydroxyethyl)terephthalate (i.e., a glycol-terephthalic acid-glycol trimer) residually remained in the white precipitate (FIG. 1, mDSC heat flow). This melting point of 109° C. is consistent with a commercially available bis(2-hydroxyethyl)terephthalate material supplied by Sigma Aldrich Co. (CAS #959-26-2). The reaction scheme outlined above and in Table 2 proved that an atmospheric pressure based system could be used to depolymerize rPET into oligomers of rPET, i.e., a resin replacement or extender composition (indicated by the 109° C. melt on the mDSC). LC-MS analysis indicated, however, that the reaction according to Table 1 had proceeded to near completion. The chemical structure and molecular weight analysis indicated that the majority of the white precipitate was actually terephthalic acid or monomer, not the rPET oligomer(s) desired. As such, the reaction conditions were modified in order to obtain greater quantities of oligomeric rPET having varying molecular weights.

The depolymerization process according to Table 2 is believed to proceed via basic hydrolysis of the ester linkages in the rPET in basic conditions using a sodium acetate catalyst. In order to obtain a low molecular weight oligomeric digested rPET species, a reaction was designed such that every additional mole of ethylene glycol (EG) added would be capable of reducing the molecular weight of the polymer through transesterification. Exemplary reactions were carried out in which 5 equivalents of ethylene glycol was added for every mole of terephthalic acid. These exemplary reactions are summarized in Table 3 and were carried out in accordance with the methods given for the initial reaction set forth in Table 2.

TABLE 3

| | Reaction 2 | Reaction 3 |
|---|---|---|
| Ethylene glycol (g) | 620 | 620 |
| PET (FW = 192 g/mol) (g) | 384 | 384 |
| Moles PET | 2 | 2 |
| ZnOAc (g) | 10 | 20 |
| Moles EG Added | 10 | 10 |

The exemplary reactions given in Table 3 were considered to be complete once the pellets of rPET were completely dissolved and the reaction reached a homogeneous, liquid phase. In each case, this required approximately 6 hours. The precipitates formed from Reaction #2 and Reaction #3 were added directly to reagent grade ethyl alcohol (Ethyl Alcohol CAS 64-17-5 90%, Methyl Alcohol CAS 67-56-1 5%, Isopropyl Alcohol CAS 67-63-0 5%) and made into a slurry. The slurries were then dried in an oven at 32° C. for 24 hrs. These resultant white products were then characterized analytically.

In each reaction, the recovered and dried precipitates comprised oligomeric units of rPET, i.e., the recovered and dried precipitates were primarily composed of incompletely digested oligomers of rPET. The mDSC analysis of the precipitates samples, according to the exemplary reactions of Table 2 (FIG. 1), showed that the material had reacted completely.

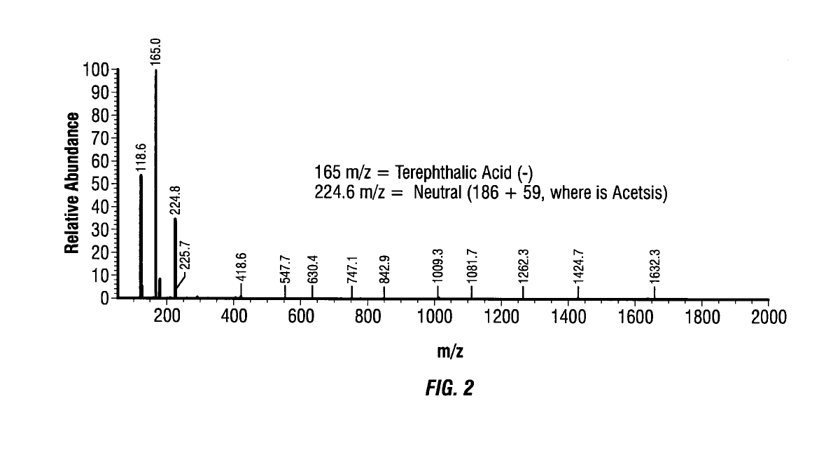

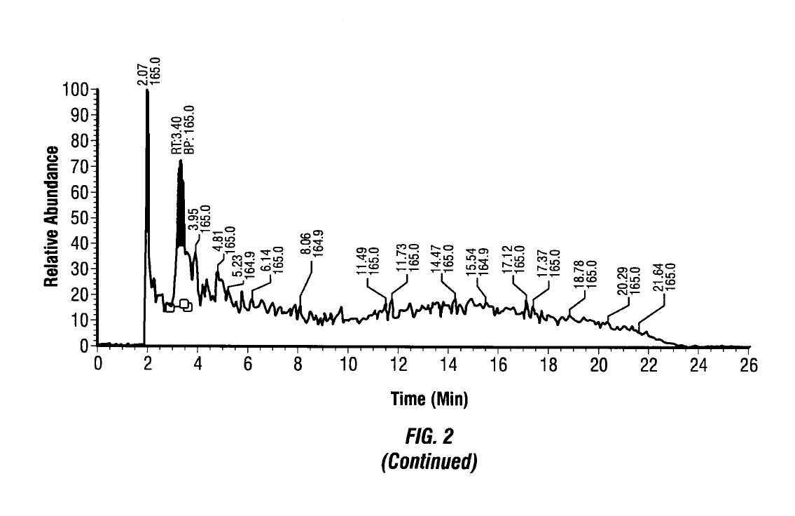

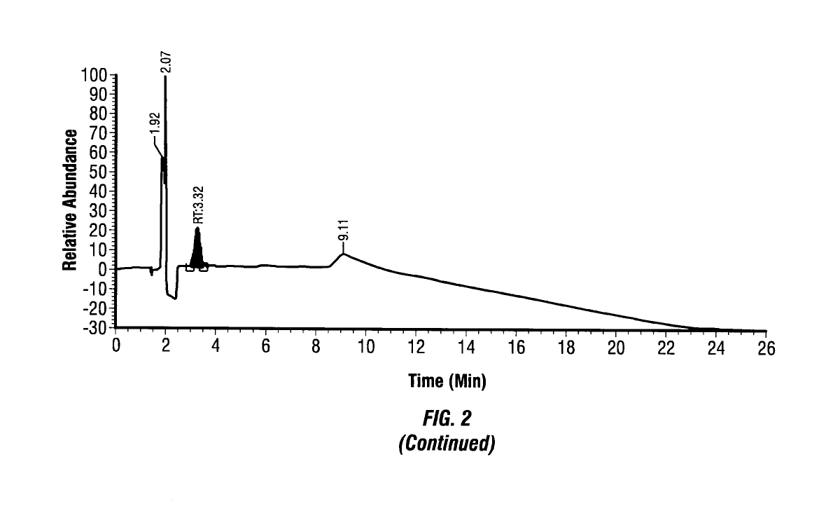

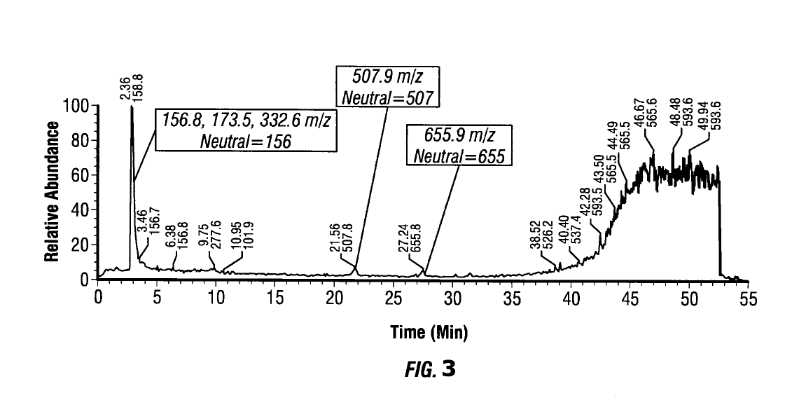

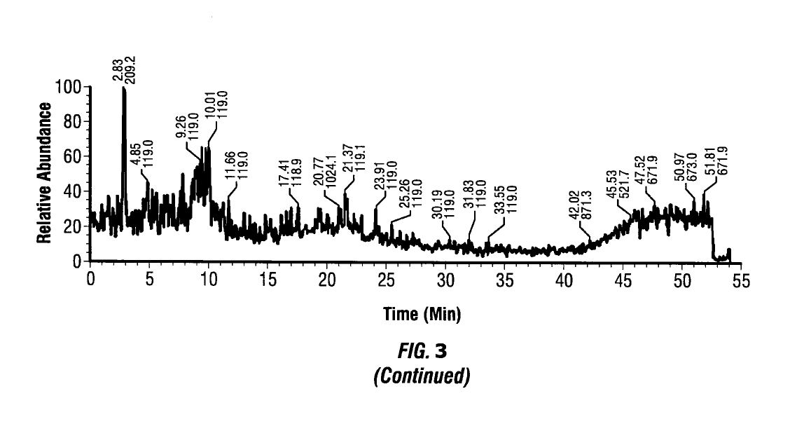

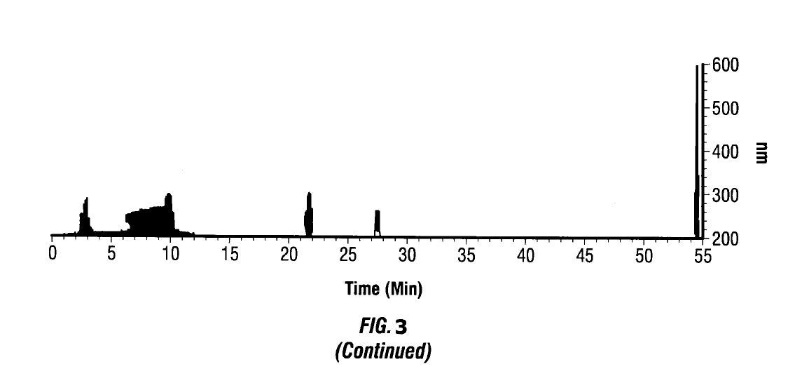

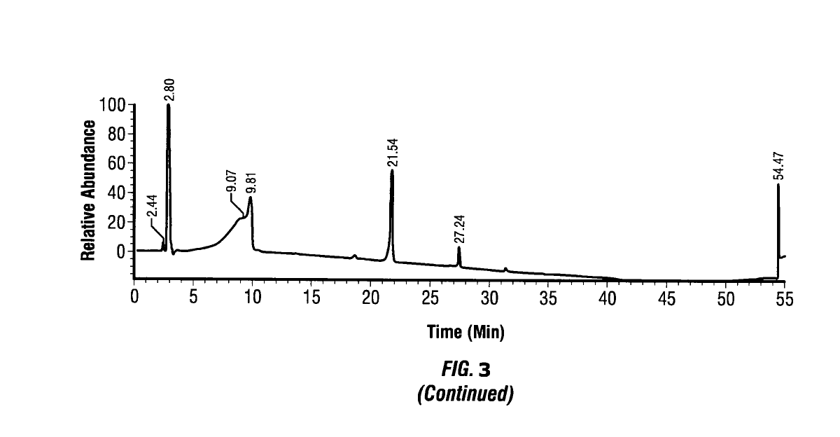

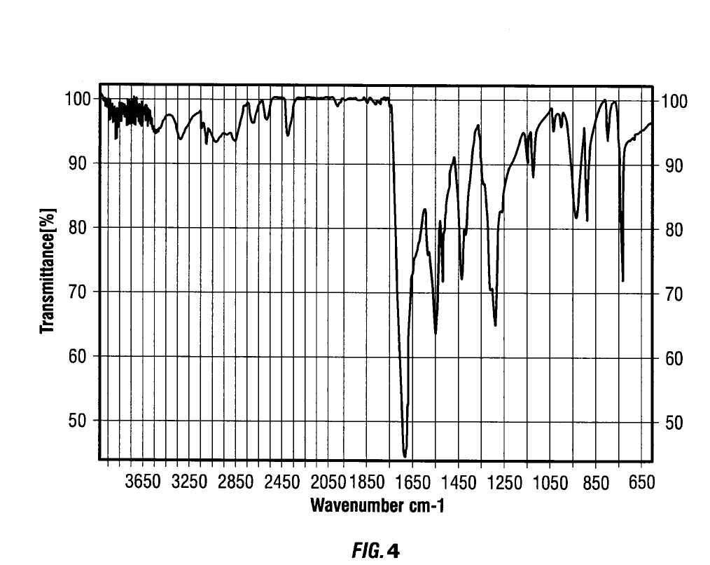

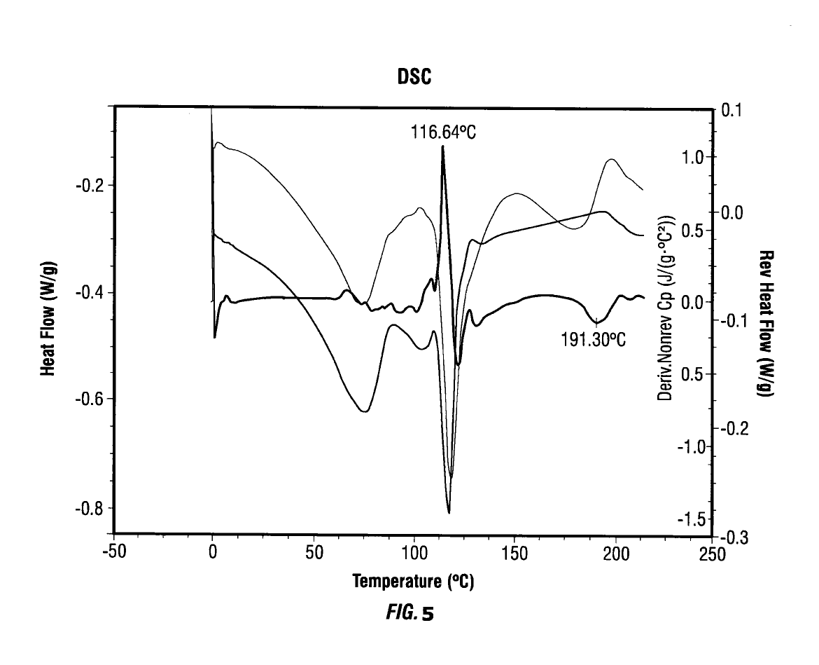

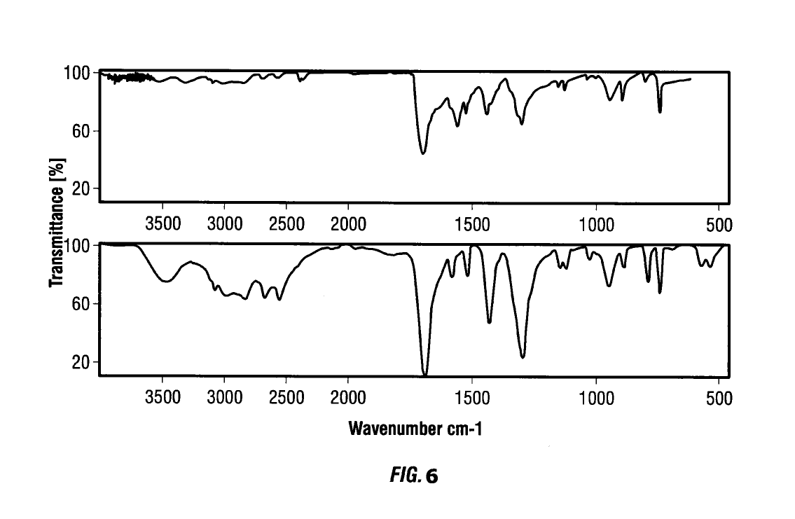

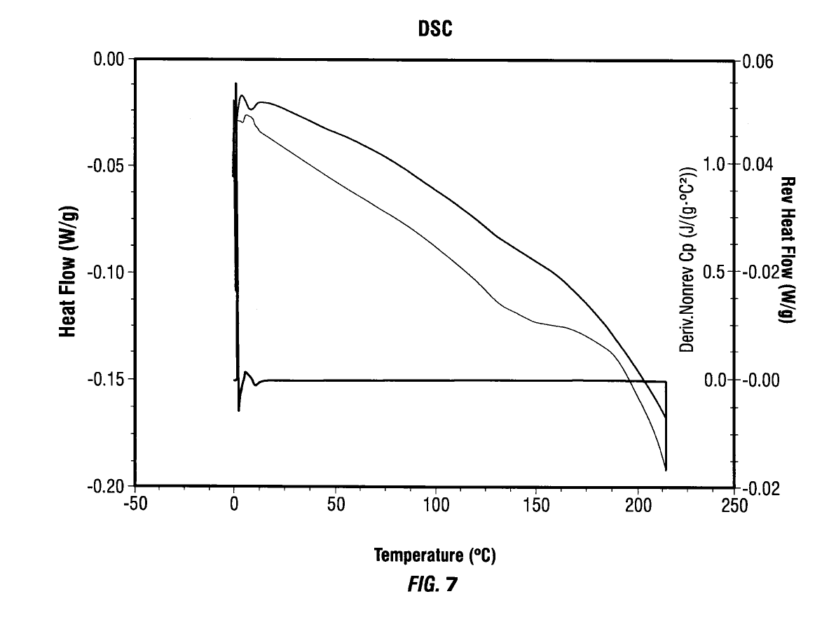

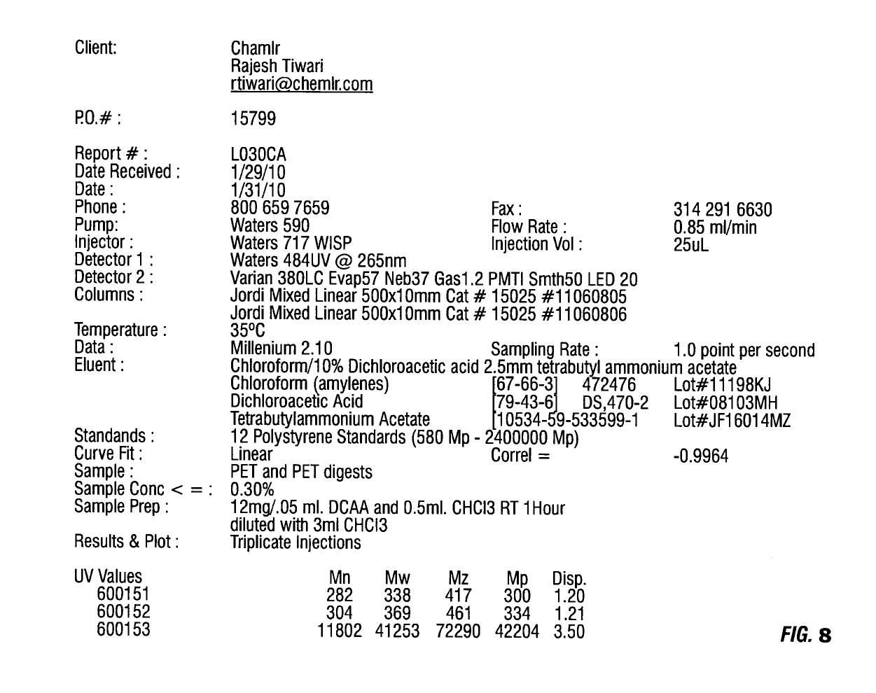

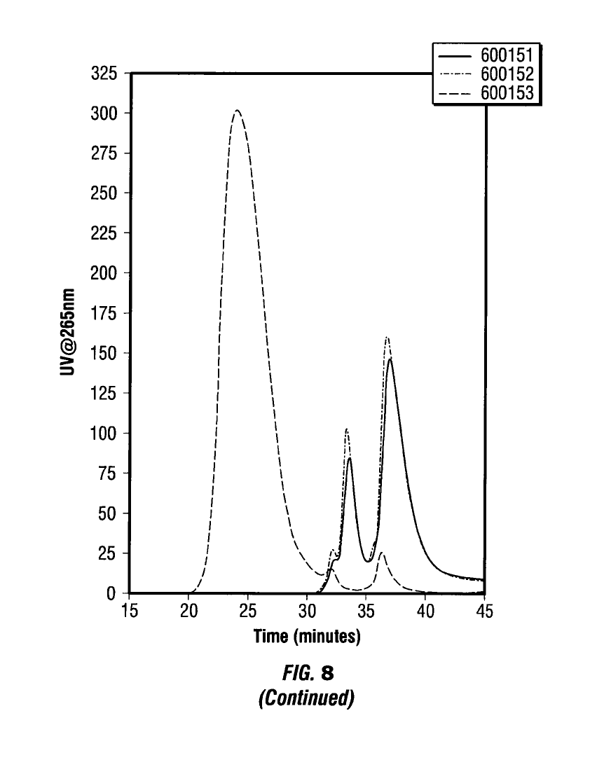

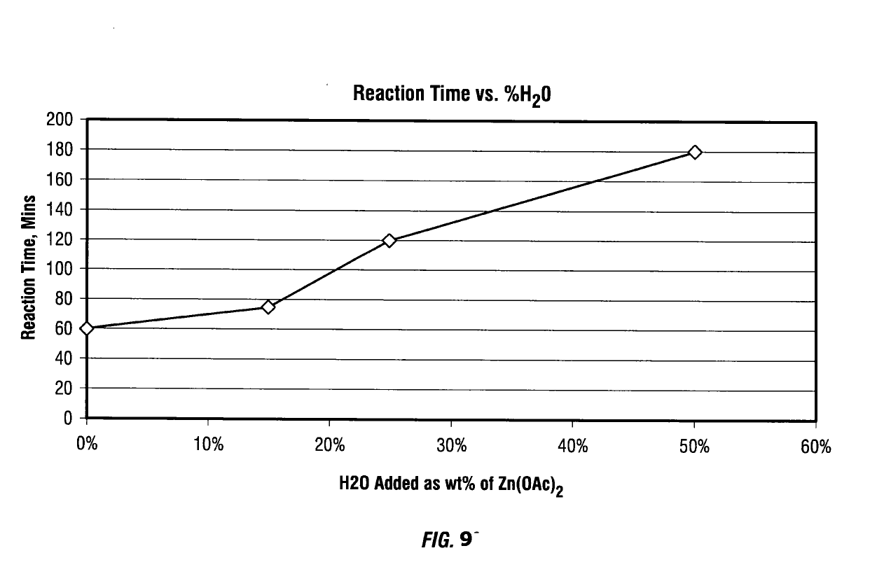

GPC characterization of the molecular weight distribution was also performed. The white product collected from these reactions was analyzed by LC/MS and Gel Permeation Chromatography to determine the molecular weight distribution of the product mixture. LC/MS analysis of the product (FIG. 2) shows three main peaks in the chromatogram. The first broad peak with a maximum at 9.81 minutes gives a mass to charge ratio consistent with the ionized forms of the trimer species. The second peak having an elution time of 21.54 minutes has a mass to charge ratio of 508. The third and smaller peak eluting at 27.24 minutes has a mass to charge ratio of 656. Although there are other peaks that have much lower UV absorbances, these are the three major, non-solvent peaks presented in the chromatograms. This analysis was repeated on three separate preparations of the material with little to no variability of the results and demonstrates that the molecular weight distribution of the material produced by these reactions is in the range of 277-656 daltons.

As is common for GPC analysis of PET, the samples were analyzed in comparison to polystyrene MW calibration standards. Duplicate preparations of the digested rPET were analyzed. The results are listed in Table 4:

TABLE 4

| | Mn | MW | Mz | Mp | Polydispersity |
|---|---|---|---|---|---|
| Undigested Scrap | 11,802 | 41,253 | 72,290 | 42,204 | 3.50 |
| PET | | | | | |
| Digested PET | 282 | 338 | 417 | 300 | 1.20 |
| Duplicate | 304 | 369 | 461 | 334 | 1.21 |
| preparation | | | | | |

As shown in Table 4, the molecular weight of the rPET has been reduced from an MW of 41,253 to an MW of 338-369. The very low polydispersity values of 1.20 and 1.21 indicate that the material is highly uniform in its molecular weight distribution. Further, the high level of agreement in the two preparations of material indicates that the procedure is repeatable and consistent. The GPC results are also in strong agreement with the LC/MS data that shows the largest peak for oligomers in the range of 277 daltons and smaller contributions for those in the 508 and 656 ranges.

Additional experiments were conducted to determine additional boundaries of the experimental design and the differentiation/production of rPET oligomers having differing molecular weights, the use of different sources and types of rPET as well as reaction kinetics and parameters. The variables studied were: (1) lowering the ratio of ethylene glycol to rPET, (2) the addition of water to the reaction, (3) differing amounts of catalyst used, (4) the use of additives, (5) the reaction time, (6) the reaction temperature, and (7) the use of mixed digestions (i.e., green and clear rPET).

While the foregoing has been described in conjunction with rPET (i.e., recycled polyethylene terephthalate), it is contemplated and has been experimentally determined (as shown hereafter) that virgin PET (vPET—i.e., polyethylene terephthalate that has not previously been molded into a product, a previously molded PET product that has not been commercially used, a previously molded PET product that has been used to hold a product or act as packaging but has not been put into commercial streams of commerce, combinations of the above, etc.) can be used in the described methods. As such, the term rPET should be understood as encompassing polyethylene terephthalate material having a recycled content of from 0% to 100% and still be within the scope of the described and claimed invention(s) herein.

Digestion of Virgin PET (Experimental Designator: 749-114): A 1 L, 4-neck flask was equipped with a mechanical stirrer, thermocouple, condenser and stopper. Neopentyl glycol (129.3 g, Aldrich 538256 lot #07304DHV) was added to the flask and melted. All of the solids dissolved when the flask was at 95° C. (internal temperature). Zinc acetate dihydrate (3.85 g, Alfa Aesar 11559, lot #C11WO13) was added in one portion. The temperature was increased to 135° C. and virgin PET (i.e., vPET—240 g, Poly Sciences 04301 lot #46418) was added in portions over a 15 min period. The temperature was raised to 200° C. and held for 4.5 h. The pellets dissolved to give a slightly hazy solution—i.e., dPET obtained from a reaction of vPET. The resulting dPET from vPET was observed to have a hydroxyl number of 354 (over an average of three determinations) which corresponds to 6.31 mmol/g, while the viscosity was measured to be 1416 centipoise (cP) at 80° C. GPC data indicated that the resulting dPET from vPET had an average MW of 1237 and the resulting chromatograph was similar to dPET from a rPET source that was digested in a similar manner. Overall, the data for virgin digested material was consistent with material prepared from recycled PET using the same stoichiometry.

### Scaling of rPET Digestion Reactions

In order to examine the ability of the rPET digestion to be scaled to commercial production scale, a series of experiments were conducted to approximate such commercial conditions.

Experimental Designator 188-73: A 22 L 4-neck flask was fitted with a Teflon stir blade connected to a high-torque overhead stirrer, thermocouple and condenser. Neopentyl glycol (3651 g, 35 mol Aldrich 538256-3KG Lot 10134519) was added to the flask and melted at 145° C. Zinc acetate dihydrate (109 g, 0.5 mol Alfa Aesar 11559 Lot A13U005) was added in portions over 2 min. Recycled PET (6743 g, 35 mol on the basis of the monomer, green pellets) were added in portions over 1 h 40 min as the set point of the temperature controller was increased to 200° C. after the final addition of rPET. The temperature was held at 200° C. until all of the pellets dissolved (approximately 4.7 h). After all of the pellets of rPET had dissolved, the solution was allowed to cool and the resulting product was packaged at approximately 72° C. Approximately 10.5 kg of digested PET product was produced.

Experimental Designator 749-74: A 22 L 4-neck flask was fitted with a Teflon stir blade connected to a high-torque overhead stirrer, thermocouple, stopper and condenser. Neopentyl glycol (3651 g, 35 mol Aldrich 538256-3KG Lot 07304DHV) was added to the flask and melted at 155° C. Zinc acetate dihydrate (109 g, 0.5 mol Alfa Aesar 11559 Lot A13U005) was added in portions over 2 min. Recycled PET (6744 g, 35 mol on the basis of the monomer, green pellets) were added in portions over 1 h 15 min. The set point of the temperature controller was increased incrementally to 200° C. The temperature was held at 200° C. until all of the pellets dissolved (approximately 3.75 h). After all of the pellets of rPET had dissolved, the solution was allowed to cool and packaged at approximately 80° C. Approximately 10.5 kg of digested PET product was produced and GPC indicated an average MW of 1389. As used herein, the term “molecular weight” or “MW” in reference to PET is defined as the peak average molecular weight (Mₚ) as determined by Gel Permeation Chromatography (GPC).

Experimental Designator Lymtal PP: A 175 gallon stainless steel reactor, fitted with a condenser, was charged with neopentyl glycol (399.4 lbs) and heated to 260° F. After the alcohol was melted, zinc acetate dihydrate (11.93 lbs) was added. After it dissolved, green recycled PET (737.7 lbs) was added in equal portions over 30 mins. The temperature was increased to 378° F. over approximately 4 h. The solids required 8 hours at 350 to 378° F. to completely dissolve. Upon cooling to 176° F., approximately 1124 lbs of digested PET product was produced and GPC indicated an average MW of 1386.

### Effect of Lowering the Ratio of Ethylene Glycol to rPET for Digestion of the rPET

A series of digestion experiments were performed to study the effect of reducing the relative amount of ethylene glycol used in the rPET digestions. Reactions were set up in 1 L round bottom flasks with a mechanical stirrer, thermocouple, temperature controller, heating mantle and condenser. The flasks were charged with rPET obtained from Clean Tech of Dundee, Mich., ethylene glycol (Aldrich “Reagent Plus” grade, the CoA indicated >99% by GC, no mention of water content in either the CoA or the Product Specification Sheet) and zinc acetate dihydrate. The reaction was performed with both clear and green rPET. The results of these experiments are summarized in Table 5.

TABLE 5

| | Molar ratio | | | | | |
|---|---|---|---|---|---|---|
| | ethylene | | | reaction | | |
| Ref. | glycol | rPET | Zn(OAc)₂ | time | | Notes |
| 185-2 | 2.79 | 1.00 | 0.014 | 1 | h | Clear PET, MP = 733 |
| | | | | | | PI = 1.111 |
| 188-7-1 | 2.12 | 1.00 | 0.014 | 30 | min | Clear PET, MP = 715 |
| | | | | | | PI = 1.131 |
| 188-1-1 | 2.14 | 1.00 | 0.014 | 1 | h | Clear PET, MP = 742 |
| | | | | | | PI = 1.129 |
| 188-5-1 | 2.01 | 1.00 | 0.014 | 35 | min | Clear PET, MP = 752 |
| | | | | | | PI = 1.132 |
| 188-10 | 1.73 | 1.00 | 0.014 | 1 | h | Clear PET, MP = 731 |
| | | | | | | PI = 1.147 |
| 188-12 | 1.73 | 1.00 | 0.014 | 5 | h | Clear PET |
| 188-9 | 1.06 | 1.00 | 0.014 | 13 | h | Clear PET |
| 188-17 | 2.79 | 1.00 | 0.014 | 57 | min | Green PET: MW 715, |
| | | | | | | 1091, 1430 |
| 188- | 1.50 | 1.00 | 0.014 | 51 | min | Green PET: MW 684, |
| 18-1 | | | | | | 1048, 1336 |
| 188- | 1.00 | 1.00 | 0.014 | 24 | min | Green PET: MW 701, |
| 18-2 | | | | | | 1082, 1386 |

The digestion experiments with clear rPET showed a general trend of longer reaction times when decreasing amounts of ethylene glycol was used. The digestion experiments with green rPET showed a general trend of a faster reaction rate with decreasing amounts of ethylene glycol.

GPC analyses were performed on the green rPET digestions. The data showed that the molecular weight distributions were consistent, regardless of whether or not clear or green rPET was used. It was also observed that the differences in the amount of ethylene glycol used in this series of reactions appeared to have a minimal impact on MW distribution. The differences in the physical characteristics of the product were more pronounced, i.e., reducing the amount of ethylene glycol produced a less waxy solid digested rPET, for example.

Further efforts towards lowering the amount of ethylene glycol required to digest the rPET were also performed. For example, the use of additives to facilitate mixing was studied. Reactions were performed with 155 g clear rPET, 30 mL ethylene glycol and either (a) 30 g of predigested rPET material from sample Ref. No. 188-5, or (b) 30 g of bis(2-hydroxyethyl)terephthalate (BHTA) as additives. Zinc acetate was added to two of the three reactions. The results are summarized in Table 6.

TABLE 6

| | molar ratio | | | | reaction |
|---|---|---|---|---|---|
| Ref. | ethylene glycol | rPET | Zn(OAc)₂ | other | time |
| 188-13 | 0.67 | 1.00 | 0.014 | 16% BHTA | 30 min |
| 188-11 | 0.67 | 1.00 | 0.014 | 16% 188-5 | 12 min |
| 188-14 | 0.67 | 1.00 | 0 | 16% 188-5 | 33 min |

Reactions were also performed with green rPET. In sample Ref. No. 188-19, the reaction pot was charged with 310 g green rPET, 60 mL ethylene glycol, 2.0 g zinc acetate dehydrate and 30 g of predigested rPET product from sample Ref. No. 188-17. The reaction time was 30 min as measured from the time the pot was >170° C. until the solids were dissolved. The results are summarized in Table 7.

TABLE 7

| | molar ratio | | | | reaction |
|---|---|---|---|---|---|
| Ref. | ethylene glycol | rPET | Zn(OAc)₂ | Other | time |
| 188-19 | 0.67 | 1.00 | 0.006 | 9.7% 188-17 | 30 min |
| 188-20 | 0.67 | 1.00 | 0.014 | 9.7% BHTA | 4.5 h |

Sample Ref. No. 188-20 was similar to sample Ref No. 188-19 except that 30 g of bis(2-hydroxyethyl)terephthalate was the additive instead of predigested rPET of Sample Ref. No. 188-17. The time for the reaction to reach completion was for Sample Ref No. 188-20 longer at 4.5 h. It is believed that the bis(2-hydroxyethyl)terephthalate chelated the Zn⁺² ions thereby increasing the time of the reaction.

These digestion experiments demonstrate that the amount of ethylene glycol used to digest the rPET material can be demonstrably lowered.

### Effect of Adding Water to rPET Digestion

The effect of adding water to the digestion of rPET was studied by running a set of reactions in 1 L round bottom flasks set up as described above for the digestion of rPET with lower amounts of ethylene glycol. The flasks were charged with rPET (obtained from Clean Tech), ethylene glycol (Aldrich “reagent plus” grade, the CoA indicated >99% by GC, no mention of water content in either the CoA or the Product Specification Sheet) and zinc acetate dihydrate. Water was added in the following amounts: 0 mL, 0.75 mL, 1.25 mL and 2.50 mL. Quantities and molar ratios of the constituents are shown in Table 8 and the results of the experiments are summarized in Table 9.

TABLE 8

| | MW | quantity, g | mol | Eq |
|---|---|---|---|---|
| Ethylene glycol | 62.07 | 192.0 | 3.09 | 1.93 |
| PET (ester), | 193 | 310.0 | 1.61 | 1.00 |
| MW = monomer | | | | |
| zinc acetate | 219.51 | 5.00 | 0.023 | 0.014 |

TABLE 9

| Ref. | % H₂O with respect to Zn(OAc)₂ | Reaction time (min) |
|---|---|---|
| 188-1-1 | 0% | 60 |
| 188-1-3 | 15% | 75 |
| 188-1-2 | 25% | 120 |
| 185-3 | 50% | 180 |

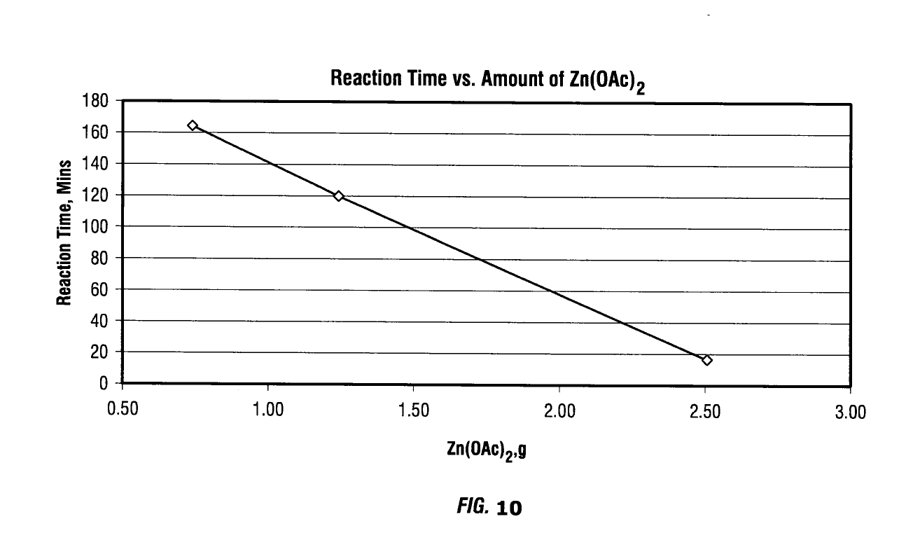

The mixtures were stirred and heated with a set point of 200° C. on the temperature controllers and full power going to the heating mantles. Reaction times were determined from the time when the pot reached a hold temperature of ca. 180-200° C. until a solution formed (solids fully dissolved). The reaction times are shown in Table 9. The data showed that the addition of water inhibited the progress of the reaction and that the greater the amount of water added, the longer the reaction time. FIG. 10 shows the reaction time for digesting rPET as a function of increased percentage water in the reaction mixture.

### Effect of Different Amounts of Zinc Acetate Catalyst on the Digestion of rPET

A series of reactions were performed with varying amounts of catalyst to determine the time necessary for reaction completion. The experiments were set up in 1 L flasks as described above with respect to the experiments determining the amount of ethylene glycol required, provided: 310 g clear rPET and 180 mL ethylene glycol. The flasks were heated to 180-195° C. with full power setting on the temperature controller. Zinc acetate (Zn(OAc)₂) was prepared as a mixture in ethylene glycol. The quantities were 2.5 g, 1.25 g and 0.75 g in 10 mL ethylene glycol and the mixtures were sonicated for 15-20 min to aid in dissolving the solids. The 2.5 g Zn(OAc)₂ preparation had some insoluble material present. The catalysts were added to the hot reaction mixtures as a bolus. Reaction times for completion, based on consumption of PET (solids fully dissolved), are summarized in Table 10. FIG. 11 shows the reaction time for digesting rPET as a function of the increased amount of catalyst (Zn(OAc)₂) in the reaction mixture.

TABLE 10

| Ref. | Zn(OAc)₂, g | time, min |
|---|---|---|
| 188-3-1 | 2.50 | 16 |
| 188-3-2 | 1.25 | 120 |
| 188-3-3 | 0.75 | 165 |

As can be appreciated from FIG. 11, a linear relationship exists between the amount of catalyst (i.e., zinc acetate) used in the reaction and the reaction time. The reaction rate appeared to be unusually fast when 2.50 g Zn(OAc)₂ was added, i.e., it was shorter than the time observed when twice the amount of catalyst was used in previous experiments. Factors that may have influenced this are the differences between temperature controllers, characteristics of the mantle or predissolving the catalyst in ethylene glycol and adding it to a hot mixture of rPET and ethylene glycol.

In order to determine if predissolving zinc acetate had any effect, the reactions described above were repeated with:
1. 5.0 g zinc acetate slurried in 10 mL ethylene glycol;
2. 1.88 g zinc acetate dissolved in 10 mL ethylene glycol; and
3. 5.0 g zinc acetate added in neat.

The reaction mixtures were heated to 180° C. and the catalyst was added. The results are summarized in Table 11.

TABLE 11

| Ref. | Zn(OAc)₂ | Reaction time, min |
|---|---|---|
| 188-5-1 | 5.00 g in 10 mL ethylene glycol | 38 |
| 188-5-2 | 1.88 g in 10 mL ethylene glycol | 130 |
| 188-5-3 | 5.00 g added neat | 65 |

The reaction time with zinc acetate slurried in ethylene glycol was almost twice as fast as what was observed when the catalyst was used neat. The reaction time for the experiment using 5.0 g of zinc acetate was still longer than that observed when 2.5 g zinc acetate was used. This can probably be attributed to differences in equipment and/or atmospheric conditions, for example. As one skilled in the art will appreciate, the reaction time decreases as the amount of catalyst increases.

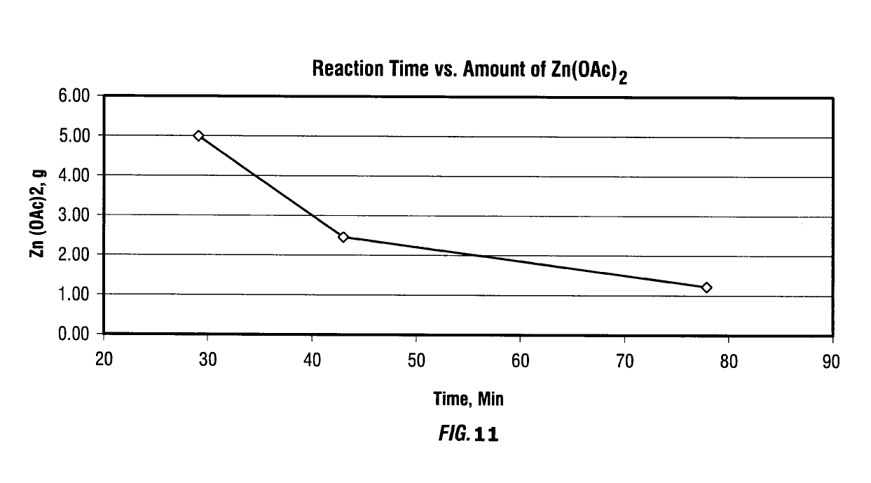

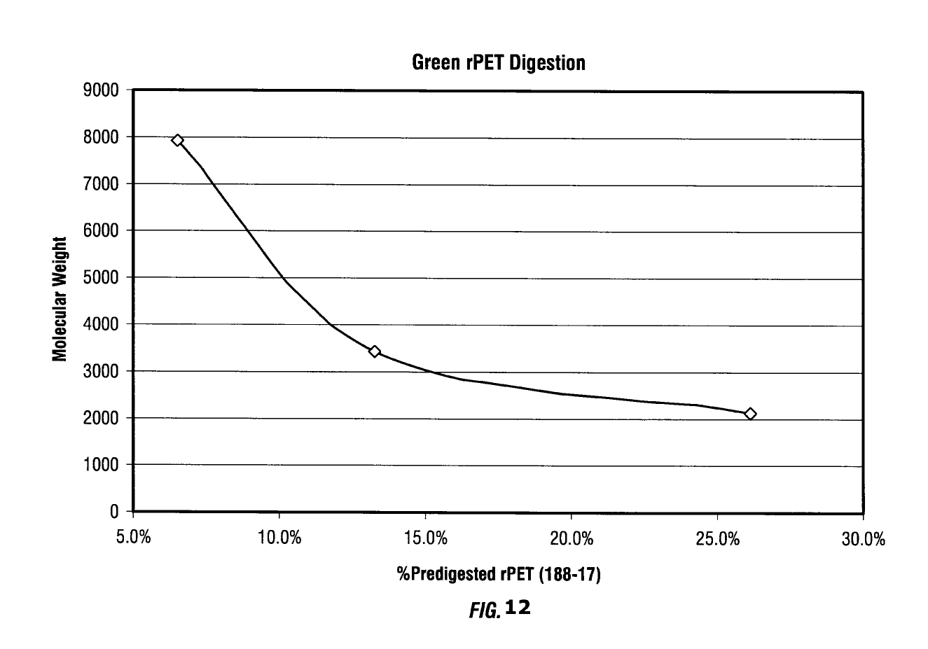

In an effort to ensure uniformity in the temperature of the pot at the start of the reaction (i.e., the addition of zinc acetate), the series was repeated by refluxing mixtures of rPET and ethylene glycol for approximately 1 h before adding the catalyst. The quantities used and the reaction times are given in Table 12. FIG. 12 graphically displays the results of reaction time versus the amount of Zn(OAc)₂.

TABLE 12

| Ref. | rPET | ethylene glycol | Zn(OAc)₂ | Reaction time, min |
|---|---|---|---|---|
| 188-7-1 | 310 g | 190 mL | 5.00 g | 29 |
| 188-7-2 | 310 g | 190 mL | 2.50 g | 43 |
| 188-7-3 | 310 g | 190 mL | 1.25 g | 78 |

In summary, it was found that decreasing the amount of catalyst resulted in longer reaction times.

### Use of Additives: Predigested PET for digestion of rPET

rPET pellets were digested with rPET that had been previously digested in earlier experiments, i.e., the previously digested portions of rPET were investigated for use as a “digesting agent” for the reactions. Using previously digested rPET appears to allow lowering the amount of ethylene glycol necessary for digesting rPET by serving as the “glycolysis” agent, free ethylene glycol can be decreased or omitted from the reaction.

In order to determine the feasibility of such a digesting agent, green colored rPET was treated with an amount of the previously digested rPET material produced in sample Ref No. 188-17. Two reactions were run with the green rPET pellets and previously digested rPET. A third reaction was run with the addition of zinc acetate to the mixture. The results of these experiments are summarized in Table 13.

TABLE 13

| | | Pre- | addi- | | reaction | | | | |
|---|---|---|---|---|---|---|---|---|---|
| | | digested | tional | | temp, | reaction | | | |
| Ref. | rPET | PET | Zn(OAc)₂ | | ° C. | time | | Mp | PI |
| 188-22 | 310 g | 50 g | 0 | | 185 | 24 | min | 3293 | 1.474 |
| 188-23 | 310 g | 50 g | 0 | | 160 | 6 | h | 3381 | 1.521 |
| 188-24 | 310 g | 50 g | 5.0 | g | 165 | 70 | min | 3743 | 1.631 |

Sample Ref. No. 188-22 was initially difficult to stir but when predigested rPET melted, mixing became easier. The reaction mixture was heated to 185° C. and went to completion in 24 min (based on rPET dissolving). Sample Ref. No. 188-23 was similar to sample Ref. No. 188-22 but run at 160-165° C. for 6 h. Mixing this reaction was difficult as a crust formed on top of the reaction mixture before all of the pellets dissolved. Therefore, it was required to be periodically broken up with a spatula. Sample Ref. No. 188-24 was similar to Sample Ref. No. 188-23 with zinc acetate added. The reaction was heated for 70 min at the end of which the liquid portion was hazy but pellets were not evident. GPC analysis of the reactions showed that the resulting digested rPET material had a molecular weight range of 3293 to 3743. Polydispersity values for the resulting material ranged from 1.474 to 1.631.

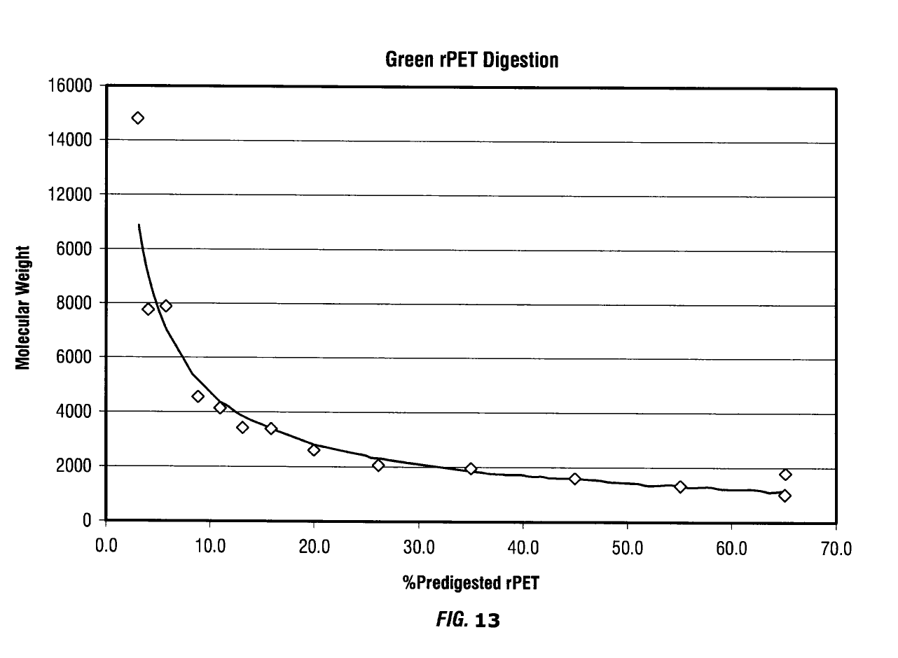

In order to produce digested rPET with different molecular weight ranges, green rPET was heated with varying amounts of previously digested rPET from Sample Ref No. 188-17 until a solution formed. The quantities used and GPC data are shown in Table 14. The relationship between the percentage of predigested rPET used (i.e., the “predigesting agent” rPET) and the molecular weight produced in the reaction showed a correlation of lower molecular weight digested rPET products being produced with increasing amounts of predigested rPET material being used as shown in FIG. 13.

TABLE 14

| | | % predigested | | |
|---|---|---|---|---|
| Ref. | Description | PET | Mp | PI |
| 188-26 | green, used predigested 188-17 | 26.1% | 2139 | 1.393 |
| 188-27 | green, used predigested 188-17 | 13.2% | 3425 | 1.519 |
| 188-28 | green, used predigested 188-17 | 6.5% | 7921 | 1.770 |

In order to further explore the range of different molecular weight products that can be produced according to the above-discussed methodology, digestions utilizing 3.3% to 65% predigested rPET were performed. Three different lots of predigested rPET were used in these experiments (all three were produced by heating a mixture of 310 g rPET with 250 mL ethylene glycol and 5.0 g zinc acetate). The experiments using varying amounts of predigested PET and GPC results are shown in Table 15 and in FIG. 13a. As shown, digested rPET having molecular weight ranges of 1058 to 14777 were produced.

TABLE 15

| | | % | | |
|---|---|---|---|---|
| | | predigested | | |
| Ref. | Description | PET | Mp | PI |
| 732-2 | green, used predigested 188-17 | 3.3 | 14777 | 1.908 |
| 733-1 | green, used predigested 188-17 | 4.3 | 7723 | 1.757 |
| 188-28 | green, used predigested 188-17 | 6.5 | 7921 | 1.770 |
| 733-4 | green, used predigested 188-17 | 9.0 | 4575 | 1.657 |
| 733-5 | green, used predigested 188-17 | 11.0 | 4151 | 1.575 |
| 188-27 | green, used predigested 188-17 | 13.2 | 3452 | 1.519 |
| 188-23 | green, used predigested 188-17 | 16.1 | 3381 | 1.631 |
| 188-29 | green, used predigested 188-17 | 20.0 | 2584 | 1.442 |
| 188-26 | green, used predigested 188-17 | 26.1 | 2139 | 1.393 |
| 733-7 | green, used predigested 734-1 | 35.0 | 1951 | 1.398 |
| 733-9 | green, used predigested 734-1 | 45.0 | 1627 | 1.345 |
| 733-11 | green, used predigested 733-10 | 55.0 | 1307 | 1.301 |
| 733-2 | green, used predigested 734-1 | 65.0 | 1058 | 1.261 |
| 732-4 | green, used predigested 188-17 | 65.2 | 1772 | 1.335 |

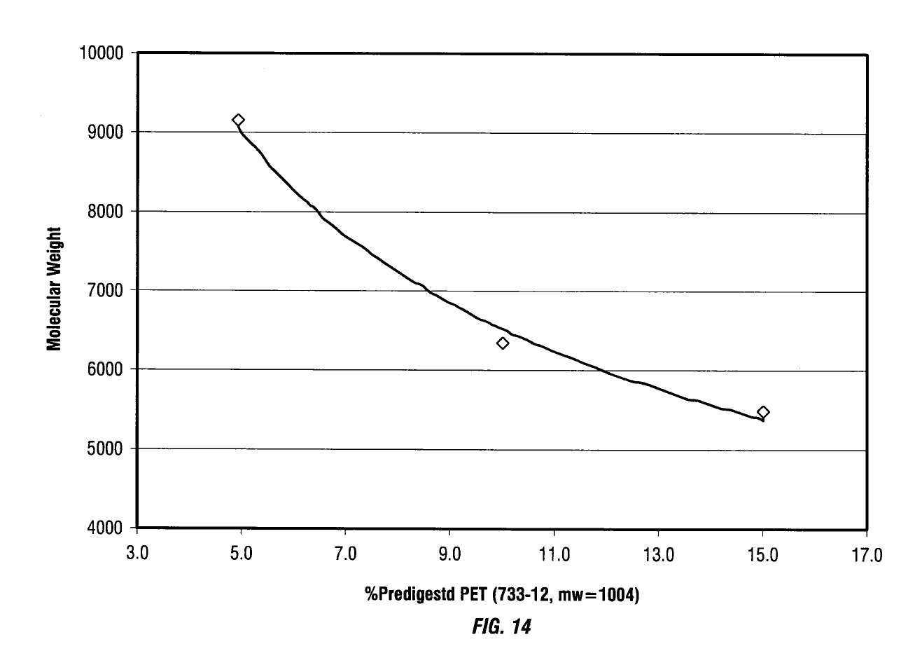

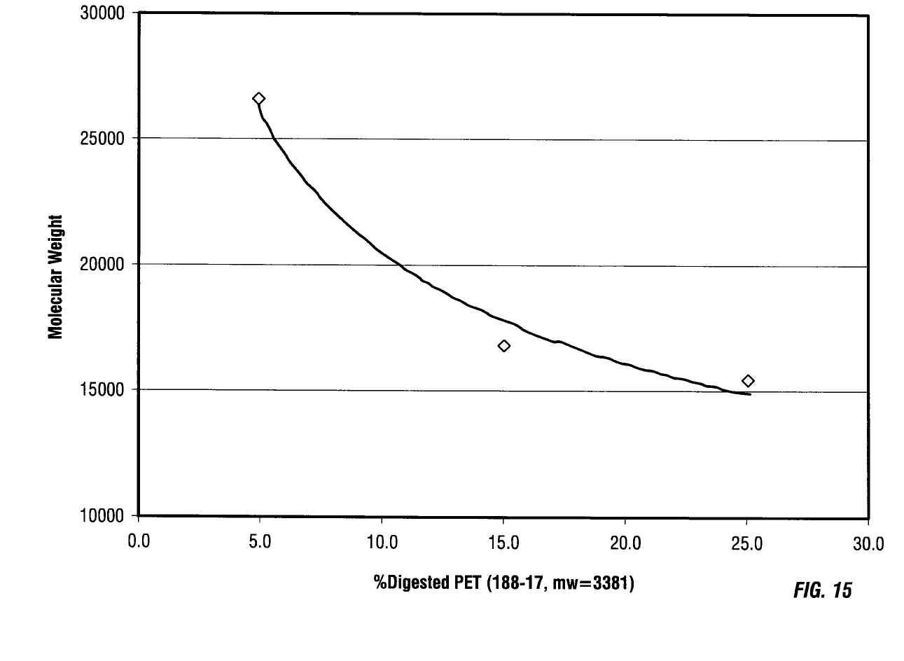

Further experiments were conducted in order to produce digested rPET having a higher molecular weight. In the first set of experiments, green rPET was heated with 5%, 10% and 15% predigested green rPET sample Ref No. 733-12 (MW 1004). In a second set of experiments, rPET was heated with 5%, 15% and 25% predigested green rPET Sample Ref. No. 188-23 (MW 3381). Both sets of experiments showed that higher molecular weight material was produced when decreasing amounts of predigested rPET material were used, e.g., using 5% predigested rPET material having a MW of 3381 produced a digested rPET material having a MW of 26,535, while 5% predigested rPET having a MW of 1004 produced a digested rPET material having a MW of 9,162. The results of these experiments are summarized in Table 16 and FIGS. 14 and 15.

TABLE 16

| | | % | | |
|---|---|---|---|---|
| | | predigested | | |
| Ref. | Description | PET | Mp | PI |
| 733-15 | green, used predigested 733-12 | 5.0 | 9162 | 1.856 |
| | (MW = 1004) | | | |
| 733-14 | green, used predigested 733-12 | 10.0 | 6342 | 1.751 |
| 733-13 | green, used predigested 733-12 | 15.0 | 5492 | 1.679 |
| 732-12 | green, used predigested 188-23 | 5.0 | 26535 | 2.261 |
| | (MW = 3381) | | | |
| 732-11 | green, used predigested 188-23 | 15.0 | 16765 | 2.133 |
| 732-10 | green, used predigested 188-23 | 25.0 | 15426 | 2.043 |

Additional experiments were also conducted with clear rPET. Clear rPET was heated with predigested rPET Sample Ref. No. 732-3 (732-3 was produced from 310 g rPET, 250 mL ethylene glycol and 5.0 g zinc acetate dehydrate, heated until a solution formed, GPC analysis of Mp=676, PI=1.103). Table 17 summarizes these results.

TABLE 17

| | | % predigested | Line | | |
|---|---|---|---|---|---|
| Ref. | Description | PET | Voltage | Mp | PI |
| 732-6 | clear, used predigest | 3.3 | 100% | 19271 | 2.148 |
| | 732-3 | | | | |
| 732-5 | clear, used predigest | 4.3 | 100% | 15176 | 2.080 |
| | 732-3 | | | | |
| 732-7 | clear, used predigest | 9.0 | 100% | 8717 | 1.809 |
| | 732-3 | | | | |
| 733-6 | clear, used predigest | 11.0 | 100% | 5348 | 1.646 |
| | 732-3 | | | | |
| 732-13 | clear, used predigest | 35.0 | 47% | 4600 | 1.639 |
| | 732-3 | | | | |
| 732-14 | clear, used predigest | 45.0 | 47% | 1598 | 1.329 |
| | 732-3 | | | | |
| 732-15 | clear, used predigest | 55.0 | 47% | 1297 | 1.306 |
| | 732-3 | | | | |

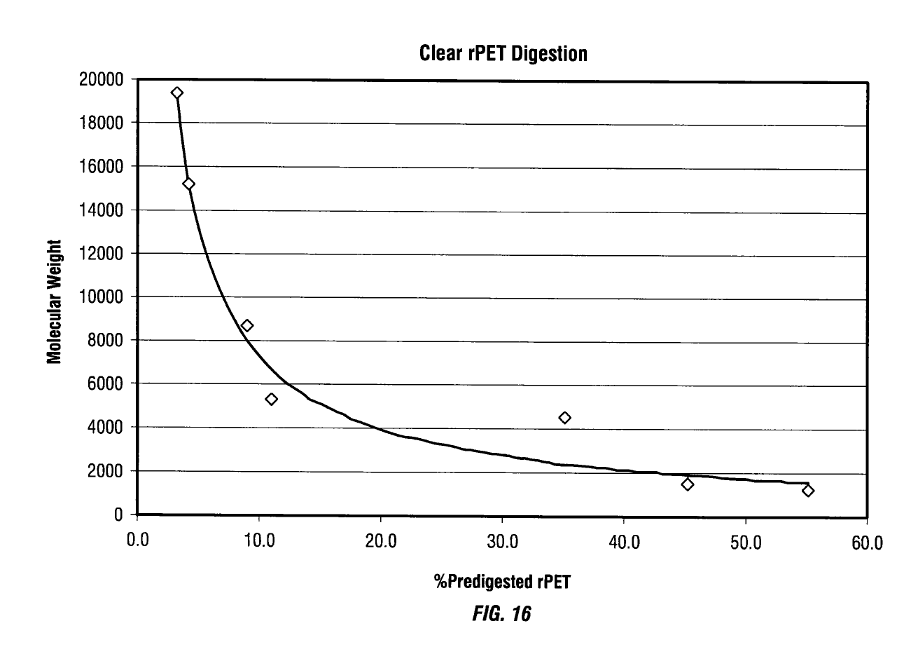

Digested rPET having a molecular weight range of 1,297 to 19,271 was produced and the polydispersity of these materials ranged from 1.306 to 2.148. FIG. 16 is a graphical representation of the percentage predigested rPET used against the molecular weight of the digested rPET produced. The curve depicted in FIG. 16 is similar to what was observed with respect to samples incorporating green rPET.

Samples with Ref Nos. 732-13, 732-14 and 732-15 were produced by reducing power to the heating source. Instead of providing 100% line voltage to the heating mantle, a variable control power transformer was used to regulate power to 47% of line voltage. (Table 17) Milder heating resulted in higher molecular weight products which is evident in the way that data points fall off the curve at union where the two different heating methods were used. Thus, treating clear rPET with decreasing amounts of predigested rPET material results in higher molecular weight digested rPET material produced.

The use of predigested rPET as a glycolysis agent has, therefore, been proved to be quite effective as a digesting agent and/or as an effective replacement for free ethylene glycol in the reaction mixture. A higher concentration of predigested rPET material gave lower molecular weight digested rPET product.

### In Situ Preparation of 16% “Predigested” rPET

Experiments were performed with both clear and green rPET to determine the effect of an in situ preparation of “predigested” rPET for use as a digesting agent.

For Sample Ref No. 732-16, 26.1 grams of clear rPET, 0.42 grams of zinc acetate and 23.4 grams of ethylene glycol were heated to reflux, achieving homogeneity at 200° C. 310.0 grams of clear rPET were added to the predigested material over 17 minutes, completing the addition at 209° C. Sample Ref No. 732-16A was taken at an internal thermocouple reading of 262° C. Fifteen minutes elapsed between completing the rPET addition and obtaining homogeneity of the mixture. GPC showed that the resulting material was digested rPET having a MP=3321 and polydispersity of PI=1.510.

For Sample Ref. No. 732-17, 26.1 grams of green rPET, 0.42 grams of zinc acetate and 23.4 grams of ethylene glycol were heated to reflux, achieving homogeneity at 195° C. 310.0 grams of green rPET were added to the pre-digested rPET material over 16 minutes, completing the addition at 199° C. Homogeneity was reached at 0.77 hours after completing the addition of the green rPET. The mixture (Sample Ref No. 732-17A) reached homogeneity at 214° C. GPC showed that the resulting material was digested rPET having a MP=3614 and polydispersity of PI=1.541.

By way of comparison, Sample Ref. No. 188-22 used 16.1% predigested rPET and produced a digested rPET material having a MP=3293 and polydispersity of PI=1.474. Thus, in situ preparation of “predigested” material gave the same results as using predigested rPET material that was isolated prior to addition to the reaction mixture.

### Effect of Reaction Time and Increased Temperature

Digestion experiments were performed to study the effect of extended reaction times and elevated temperatures of the digested rPET produced. Reactions with extended reaction times were carried out with predigested rPET mixed with rPET pellets as described above but the reaction was held at 190-200° C. for up to 6 h beyond completion. The starting point (t=0) was determined when the rPET pellets went completely into solution. The reaction mixtures were sampled at regular intervals and the pulls were analyzed by GPC.

Tables 18 and 19 summarize the concentrations of predigested rPET which were used and the results of the GPC analysis. When 9% to 65% predigested rPET were used, a long reaction time did not make a difference in the molecular weight of the digested rPET produced (within the limits of the technique). At low concentrations, longer reaction times resulted in lower molecular weight digested rPET products. For instance, the experiment with 3.3% predigested rPET produced a digested rPET product having a MW=19,271 at t=0 and MW=15,407 at t=6 h; the experiment with 4.3% predigested rPET produced a digested rPET product having a MW=15,176 at t=0 and MW=12,854 at t=5.5 h.

TABLE 18

| Ref. | Description | Mn | MP | MW | PI |
|---|---|---|---|---|---|
| 733-2A | Green PET digested with 65% | 1162 | 1058 | 1466 | 1.261 |
| | predigested PET (188-17) t = 0 h | | | | |
| 733-2B | t = 1 h | 1162 | 1053 | 1464 | 1.260 |
| 733-2C | t = 2 h | 1160 | 1050 | 1464 | 1.262 |
| 733-2D | t = 3 h | 1170 | 1053 | 1476 | 1.261 |
| 733-2E | t = 4 h | 1146 | 1034 | 1447 | 1.264 |
| 733-2F | t = 5 h | 1137 | 1021 | 1438 | 1.264 |
| 733-2G | t = 6 h | 1133 | 1015 | 1431 | 1.263 |
| 733-4A | Green PET digested with 9% | 3352 | 5185 | 5646 | 1.684 |
| | predigested PET (188-17) t = 5 h | | | | |
| 733-4B | t = 4 h | 3286 | 5106 | 5490 | 1.671 |
| 733-4C | t = 3 h | 3229 | 4917 | 5321 | 1.648 |
| 733-4D | t = 2 h | 3188 | 4780 | 5239 | 1.643 |
| 733-4E | t = 1 h | 3074 | 4612 | 5073 | 1.650 |
| 733-4F | t = 0 h | 3014 | 4575 | 4994 | 1.657 |
| 733-4G | t = 6 h | 3342 | 5287 | 5636 | 1.686 |

TABLE 19

| Ref. | Description | Mn | MP | MW | PI |
|---|---|---|---|---|---|
| 732-6A | Clear PET digested with 3.3% | 9513 | 19271 | 20430 | 2.148 |
| | predigested PET (732-3) | | | | |
| | t = 0 h | | | | |
| 732-6B | t = 1 h | 9580 | 19169 | 21590 | 2.254 |
| 732-6C | t = 2 h | 9248 | 18779 | 21497 | 2.324 |
| 732-6D | t = 3 h | 9357 | 18374 | 21510 | 2.299 |
| 732-6E | t = 4 h | 8813 | 16739 | 21010 | 2.384 |
| 732-6F | t = 5 h | 8506 | 15823 | 20391 | 2.397 |
| 732-6G | t = 6 h | 8254 | 15407 | 20137 | 2.440 |
| 732-5A | Clear PET digested with 4.3% | 7785 | 15176 | 16190 | 2.080 |
| | predigested PET (732-3) | | | | |
| | t = 0 h | | | | |
| 732-5B | t = 0.5 h | 7839 | 14627 | 15643 | 1.996 |
| 732-5C | t = 1.5 h | 8359 | 16932 | 19093 | 2.284 |
| 732-5D | t = 2 h | 8403 | 16625 | 19605 | 2.333 |
| 732-5E | t = 2.5 h | 8237 | 16561 | 19796 | 2.404 |
| 732-5F | t = 3.75 h | 7669 | 15013 | 19189 | 2.502 |
| 732-5G | t = 4.75 h | 7201 | 14236 | 18899 | 2.624 |
| 732-5H | t = 5.5 h | 6945 | 12854 | 17562 | 2.529 |
| 733-5A | Clear PET digested with 11% | 2848 | 4151 | 4484 | 1.575 |
| | predigested PET (732-3) | | | | |
| | t = 0 | | | | |
| 733-5B | t = 1 h | 2882 | 4178 | 4527 | 1.570 |
| 733-5C | t = 2 h | 2864 | 4261 | 4568 | 1.595 |
| 733-5D | t = 3 h | 2896 | 4332 | 4620 | 1.595 |
| 733-5E | t = 4 h | 2923 | 4478 | 4736 | 1.620 |
| 733-5F | t = 5 h | 2971 | 4482 | 4821 | 1.623 |
| 733-5G | t = 6 h | 2989 | 4618 | 4915 | 1.644 |

The effect of elevated reaction temperatures was further analyzed by performing a digestion reaction with 100.0 grams of clear rPET with 3.3% predigested clear rPET. Homogeneity was achieved 2.92 hours from the time that heat was applied to the clear rPET. The longer time required to reach homogeneity was a function of the attempt to control the temperature and heating rate. Three samples of the homogeneous mixture were taken and GPC results tabulated:
1. 216° C. internal temperature (Ref. No. 732-18A, time 1235), MP=21171 PI=2.088
2. 249° C. internal temperature (Ref. No. 732-18B, time 1419), MP=19616 PI=2.185
3. 272° C. internal temperature (Ref. No. 732-18C, time 1530), MP=15459 PI=2.231

Thus, reaction time has little, if any, effect on the molecular weight of the product when concentrations of predigested rPET are >9%. Low concentrations of predigested rPET, however, indicate that the molecular weight of the digested rPET product decreased as time passed.

Increasing the reaction temperature resulted in lower molecular weight products. Although additional time was needed to raise the temperature of the reaction mixture, the additional time needed was 3 h. The additional time was most likely not sufficient alone to be a factor in the observed results.

### Mixed Green and Clear rPET Digestions

Digestion experiments were performed with a mixture of green and clear rPET to determine the effect of blending these recycled materials, i.e., to determine whether the separation of green and clear rPET is necessary for the digestion reactions to occur. Sample Ref No. 733-18 is summarized in Table 20; 1:1 mixture of green and clear PET was digested with ethylene glycol and zinc acetate using stoichiometry which produced material used for most of the predigestion experiments. The reaction behaved normally. GPC showed that the resulting material was a digested rPET having a MP=701 and polydispersity of PI=1.112.

TABLE 20

| | | | | | reaction |
|---|---|---|---|---|---|
| Ref. | ethylene glycol | green rPET | clear rPET | Zn(OAc)₂ | time |
| 733-18 | 250 mL | 155 g | 155 g | 5.00 g | 93 min |

The product from above was then used as a predigested rPET starting material in the digestion of 1:1 mixtures of green and clear rPET, and the experiments are summarized in Table 21. GPC results showed that the resulting digested rPET material had a very low polydispersity despite the fact that the clear and green rPET have different average molecular weights.

TABLE 21

| | | % predigested | | |
|---|---|---|---|---|
| | | PET (Ref. No. | | |
| Ref. | Description | 733-18) | MP | PI |
| 733-19 | 155 g green PET + 155 g clear | 15.0% | 3546 | 1.547 |
| | PET + 733-18 | | | |
| 733-20 | 155 g green PET + 155 g clear | 25.0% | 2380 | 1.421 |
| | PET + 733-18 | | | |
| 733-22 | 155 g green PET + 155 g clear | 55.0% | 1120 | 1.228 |
| | PET + 733-18 | | | |

It has been shown, therefore, that recycled PET (rPET) can be converted to lower molecular weight polymers by glycolysis with ethylene glycol and zinc acetate. Material which was digested in this manner can thereby become a digestion agent to produce a wide range of molecular weight polymers from rPET starting material. Low concentrations of predigested rPET used as the “starter” gave high molecular weight products while, conversely, high concentrations of predigested rPET used as the “starter” gave low molecular weight products. Experiments with green colored rPET and clear colored rPET behaved similarly to experiments where green and clear colored rPET where mixed together.

The precipitates from each of the reactions, i.e., the digested rPET oligomers, were then each separately tested as to suitability as a resin replacement and/or resin extender.

### Removal of Green Tint Remaining in Digested Green rPET

Initially, pre-digested green PET was reacted with 2,2-dimethyl-1,3-propane diol to make higher molecular weight polymers. In particular, 250 mL of toluene were brought to reflux in a 4-neck reactor fitted with a Dean-Stark trap. 125.0 grams of predigested green rPET from Sample No. 732-34 (Table 22 below) were added in portions to the refluxing toluene. It appeared that the refluxing toluene could accept more than the 125.0 grams of digested rPET material.

The homogeneous green reaction mixture was heated at reflux (111.0-113.1° C.) for 6 hours after completing the digested rPET addition. 14.6 mL of water was collected from the Dean-Stark trap. As the reaction mixture cooled through 102.7° C., a 2-phase mixture formed on stopping the agitator, an upper, homogeneous green layer and a lower, opaque faint green layer. At approximately 75° C. the mixture began to solidify. After cooling to ambient temperature, the heterogeneous mixture was transferred to a beaker and allowed to stand at ambient temperature overnight. Decanted homogeneous green solution was used to complete the transfer to the beaker.

Vacuum filtration of the mixture produced a white filter cake having a faint blue-green tint and a lime green homogeneous filtrate. The filter cake was washed with 50 mL of toluene. The filter cake yielded 105.26 grams of white solid material after drying the solid on a 40-50° C., Buchi pump rotary evaporator for 15 minutes followed by 15 minutes on a 40° C., 0.1 mm Hg vacuum Kugelrohr. 0.32 grams of green semi-solid were recovered from the concentration of 50 mL of the green filtrate on a rotary evaporator at 40-50° C. and Buchi pump vacuum followed by 15 minutes on a 40° C., 0.4 mm Hg vacuum Kugelrohr.

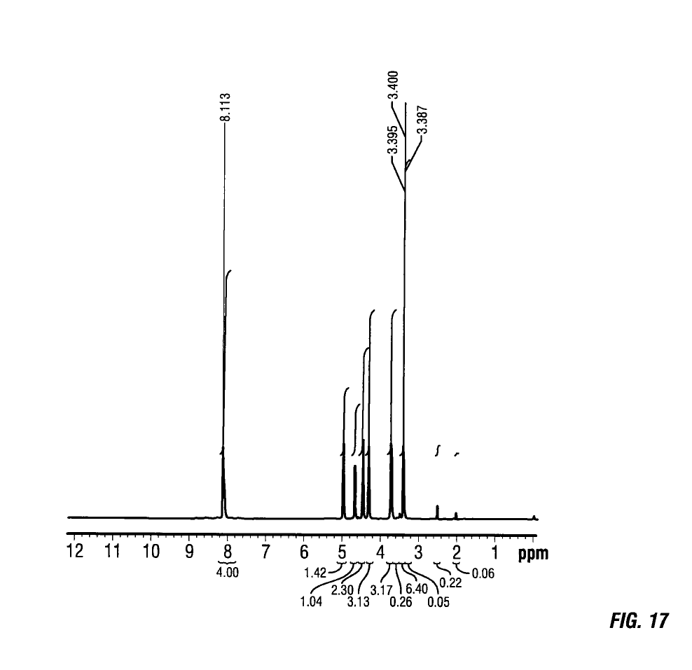

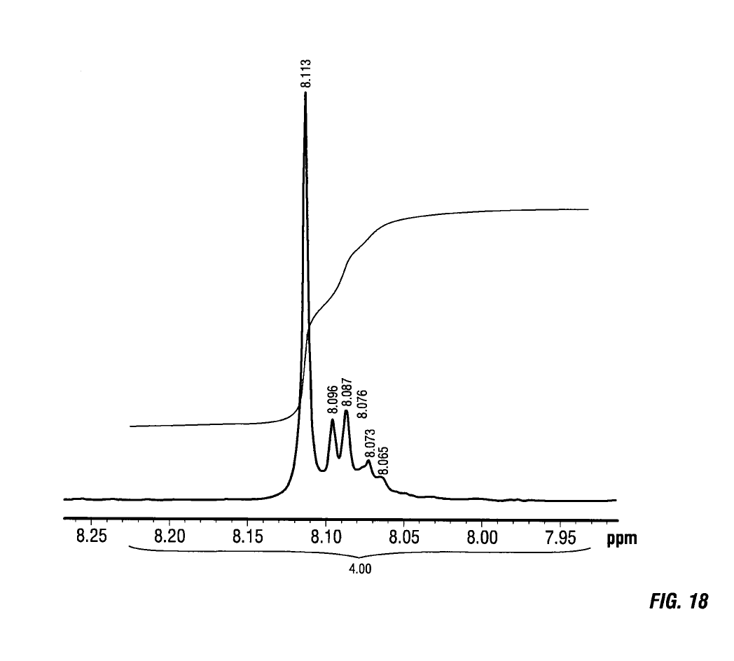

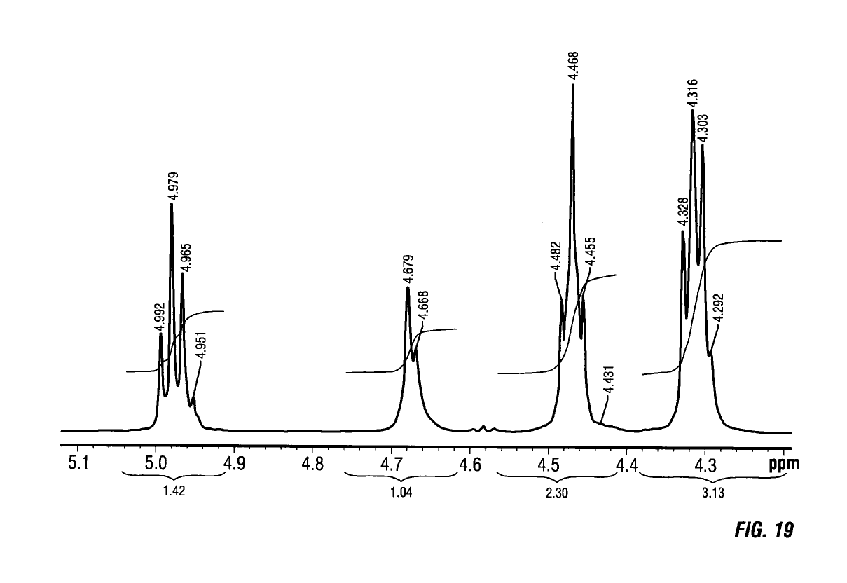

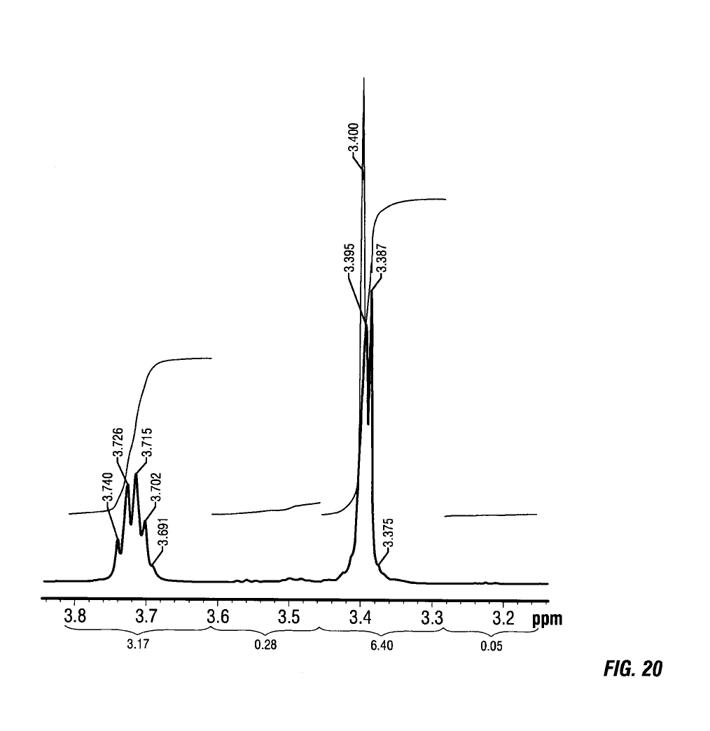

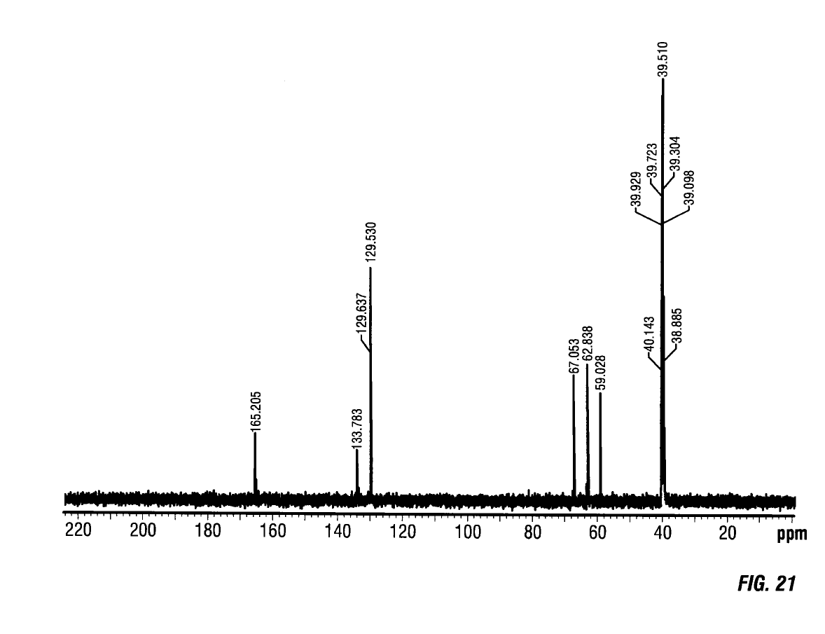

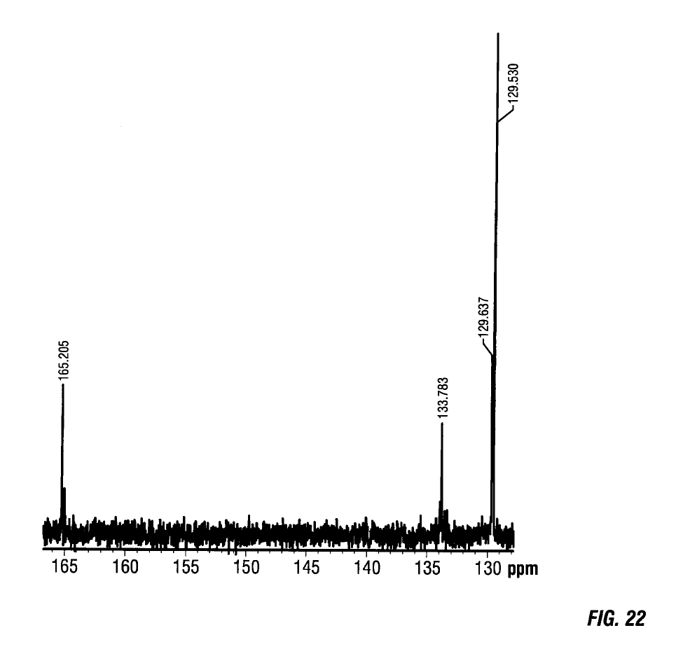

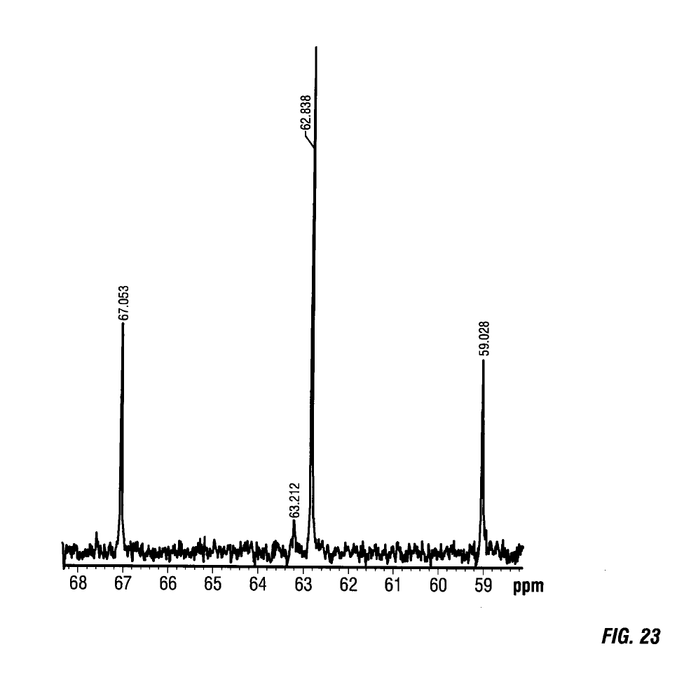

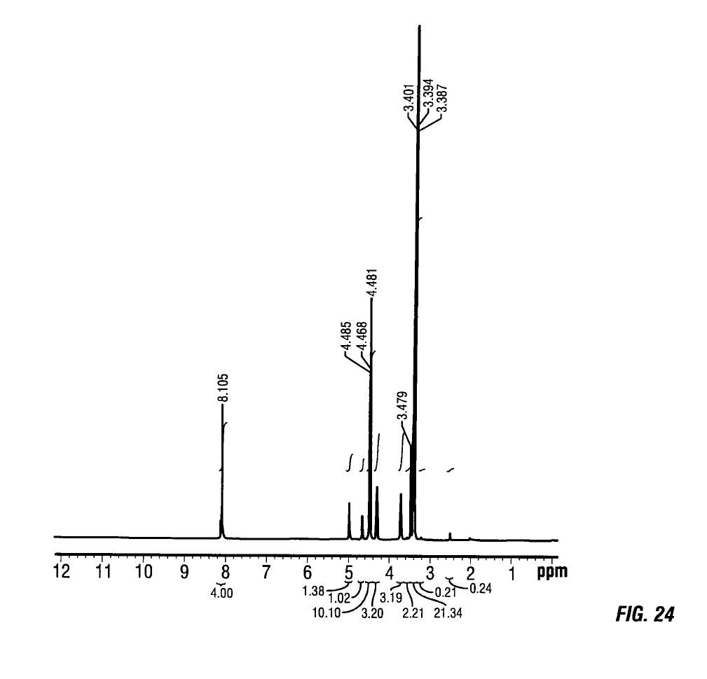

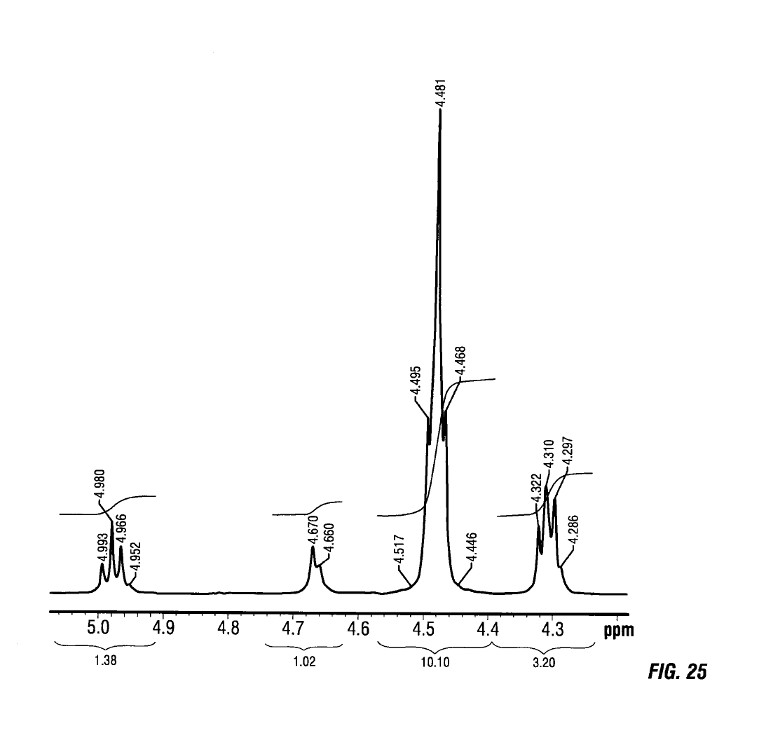

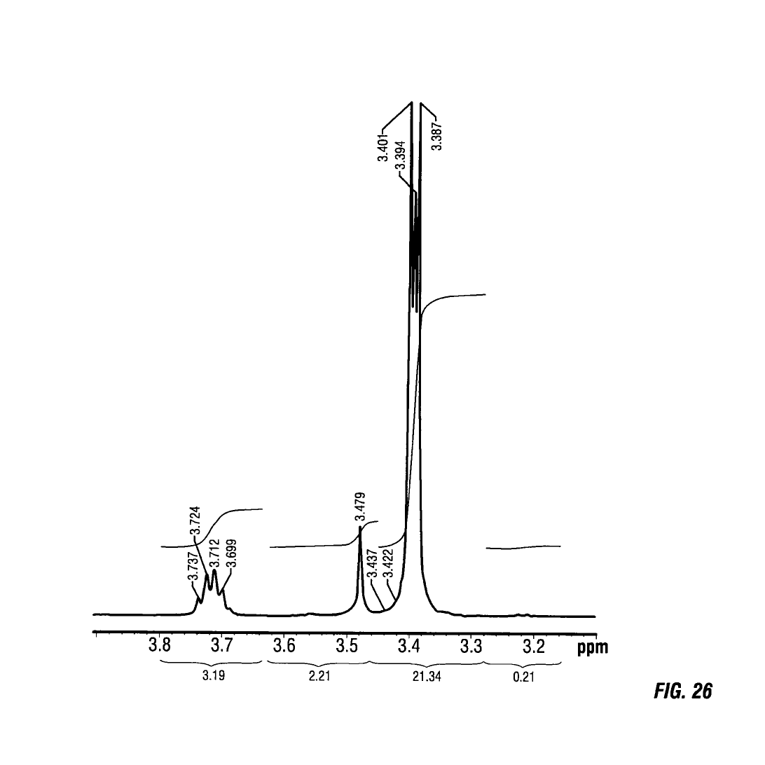

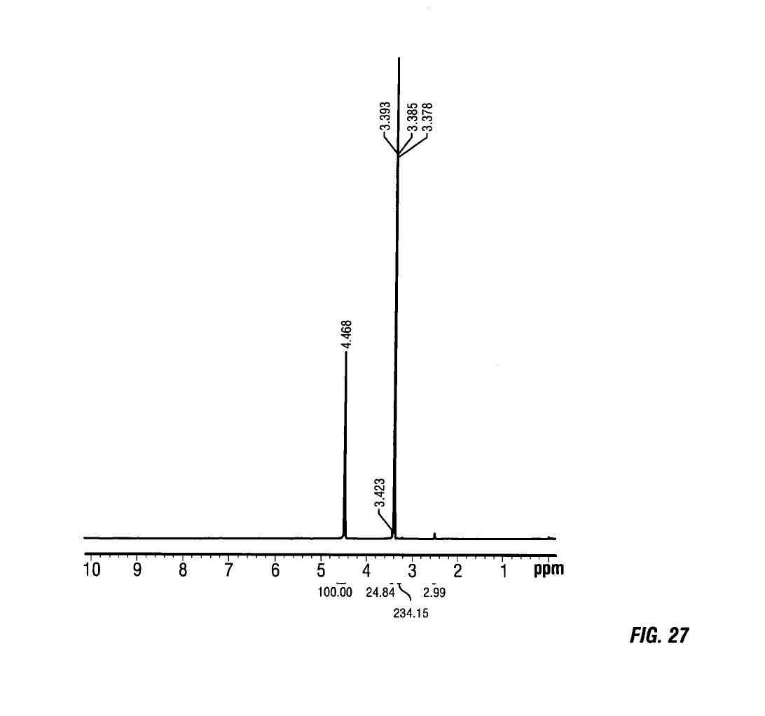

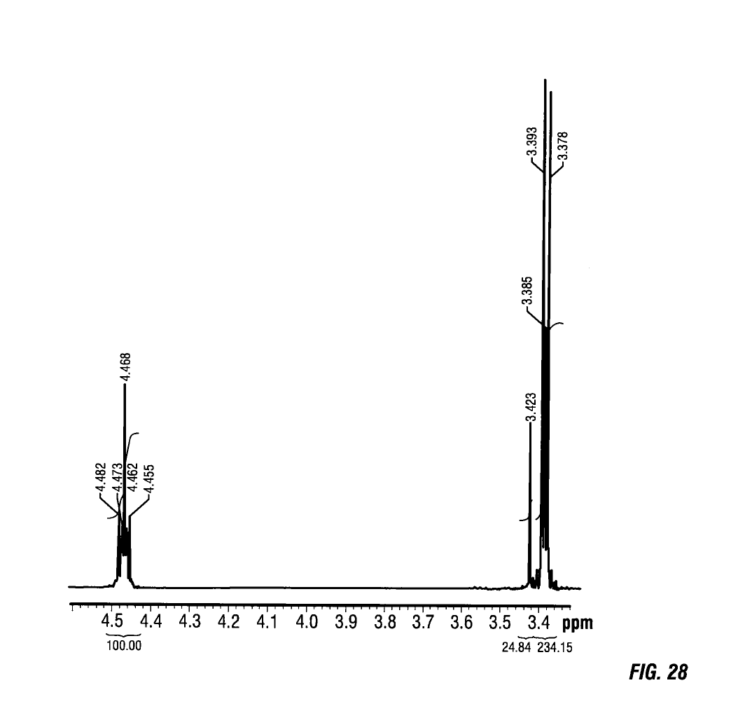

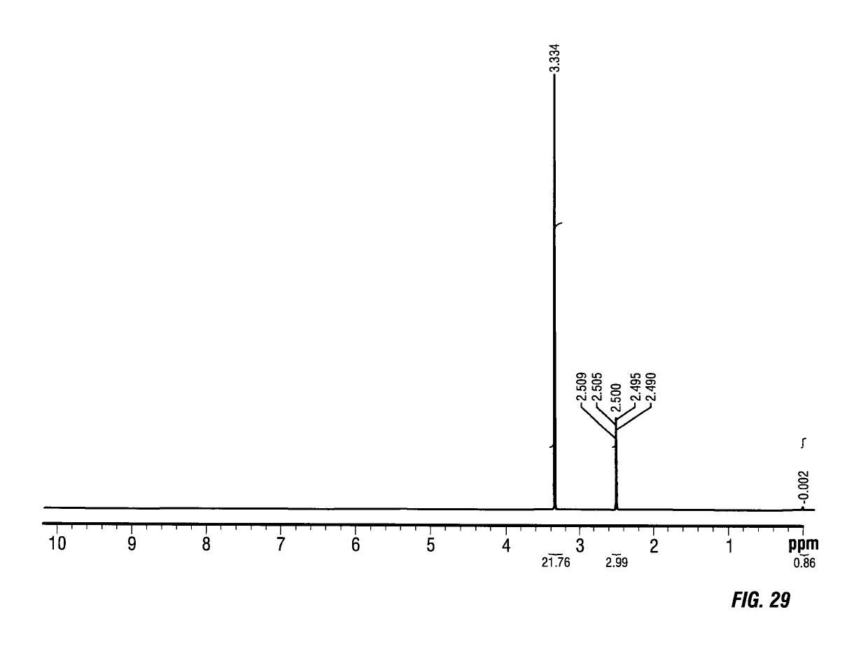

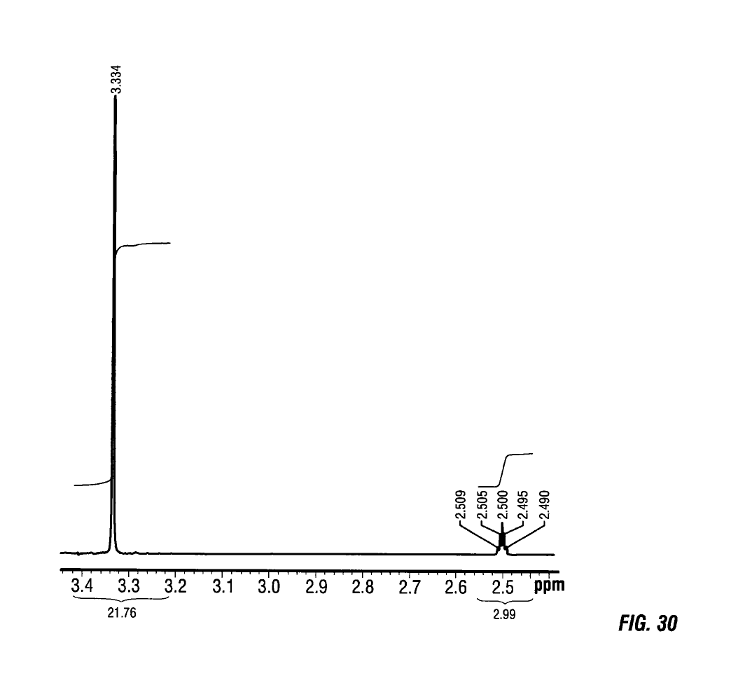

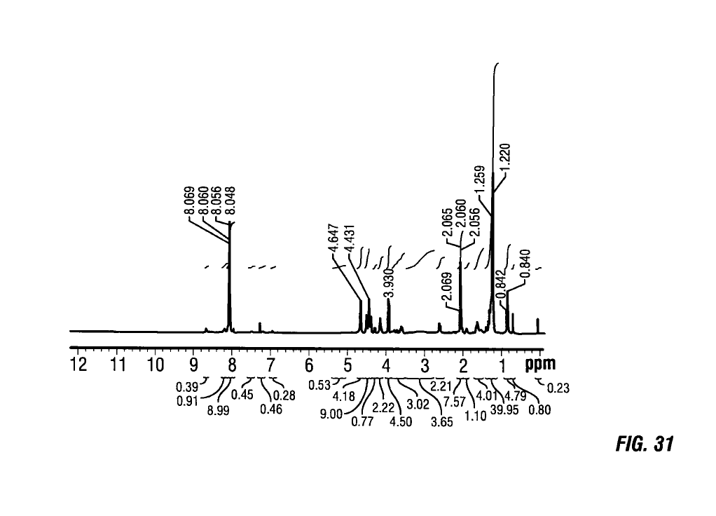

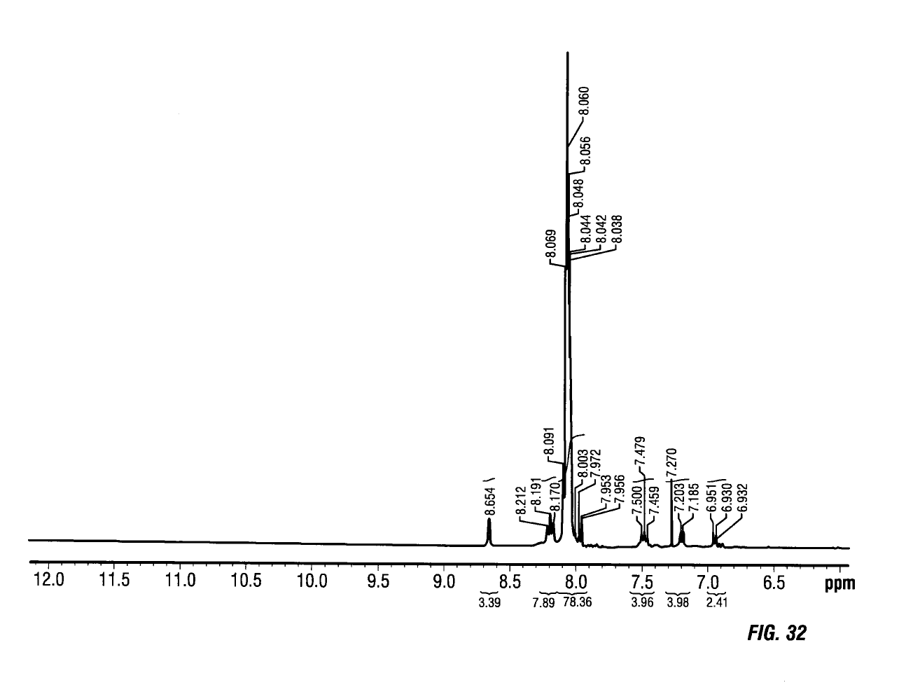

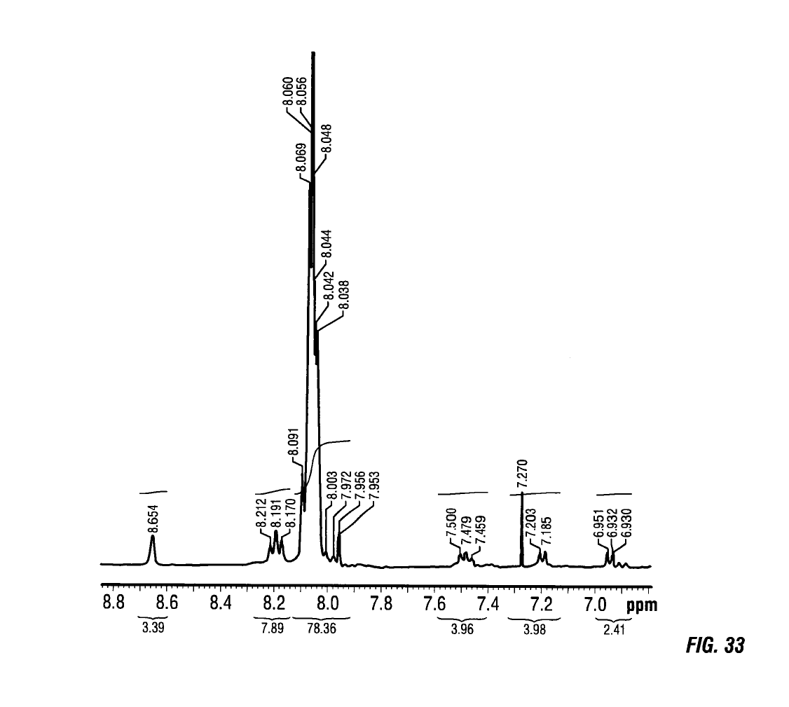

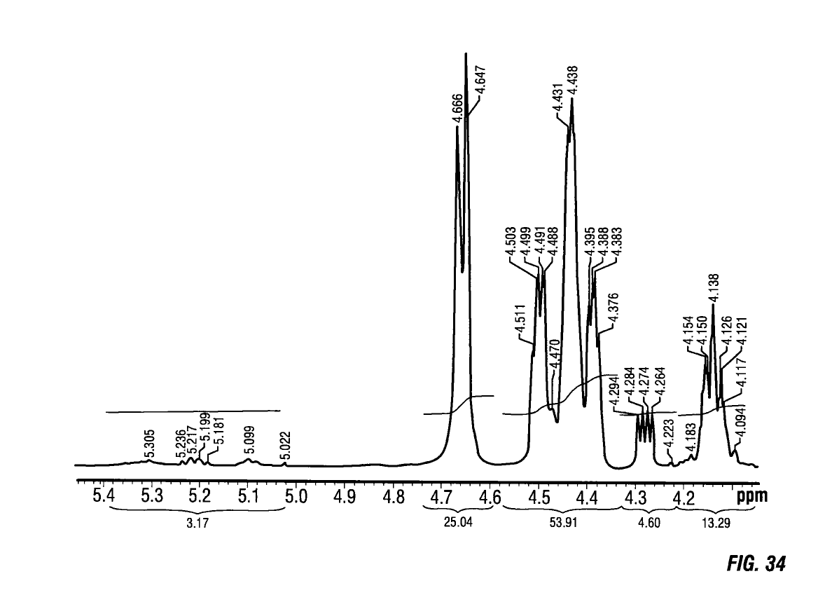

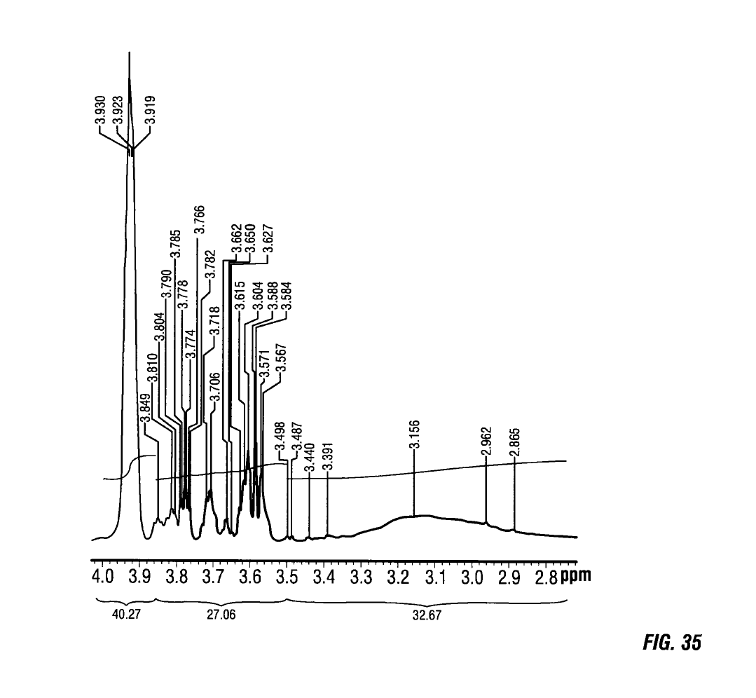

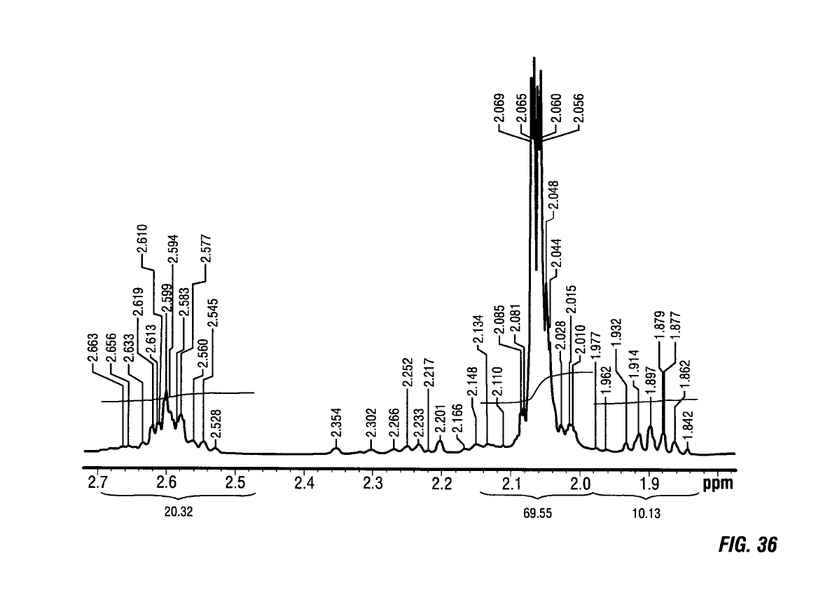

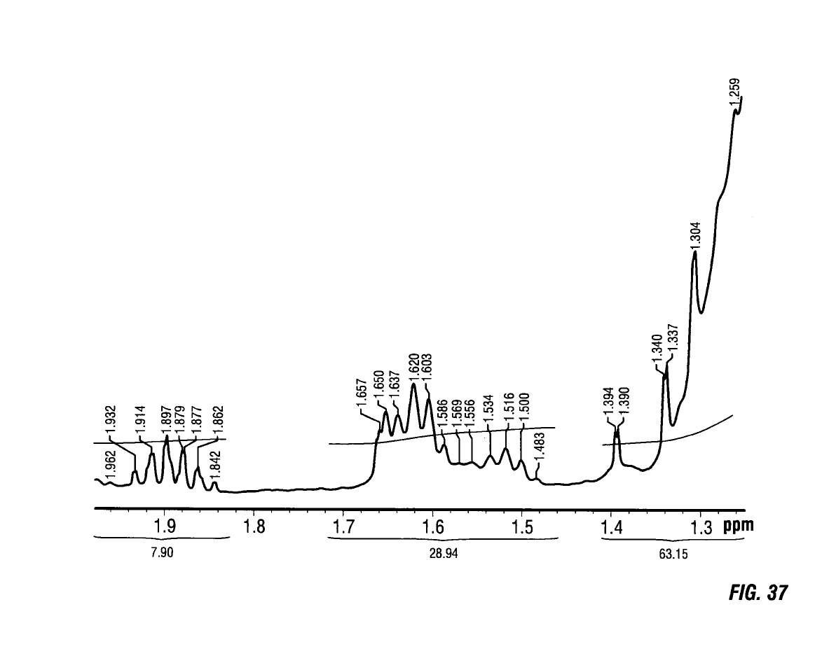

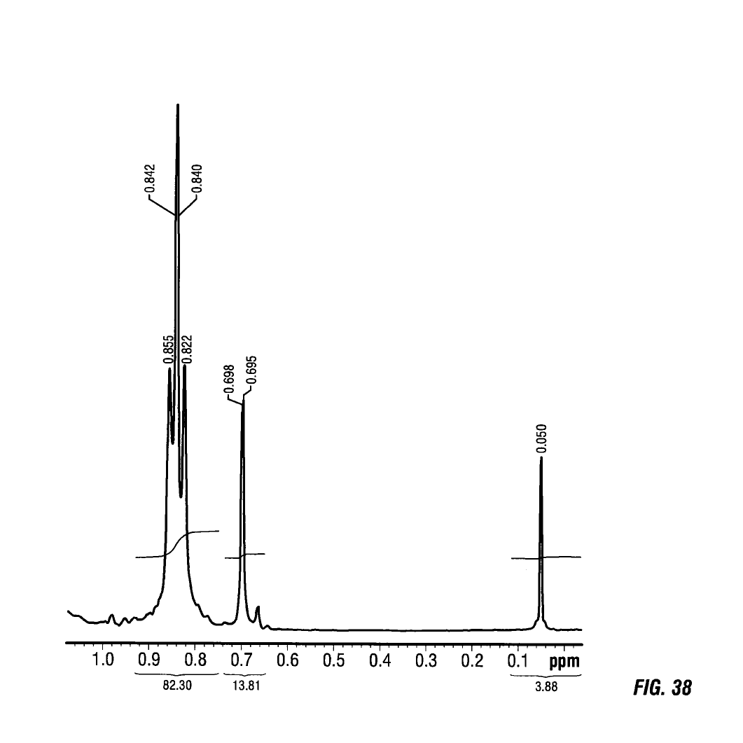

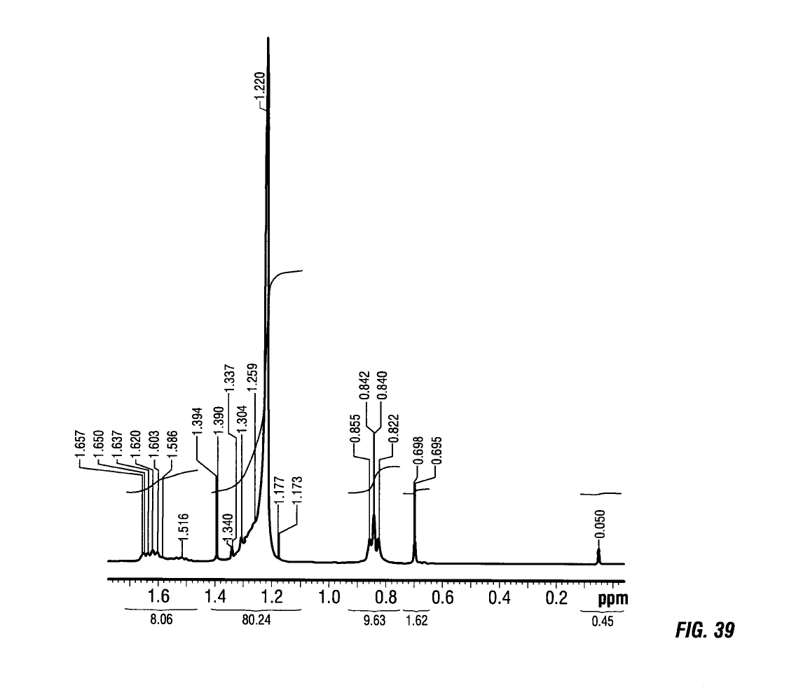

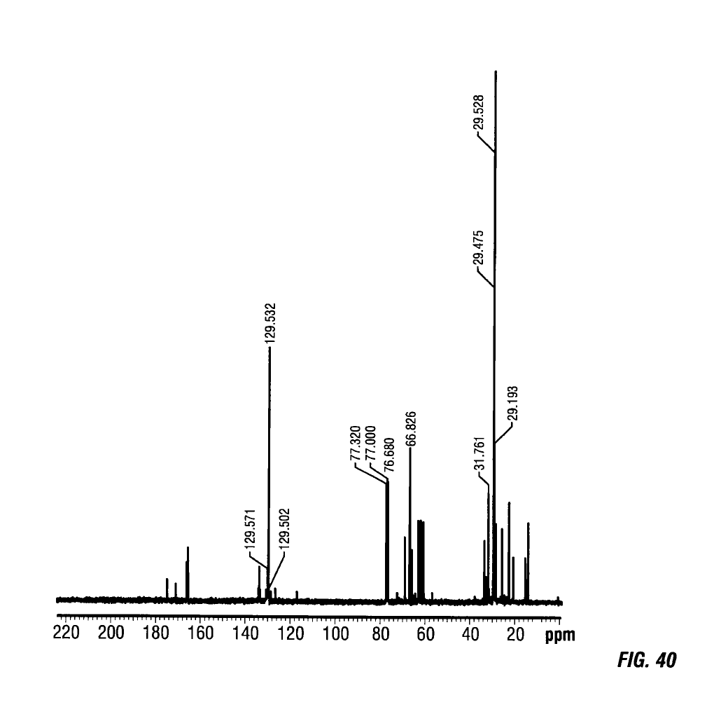

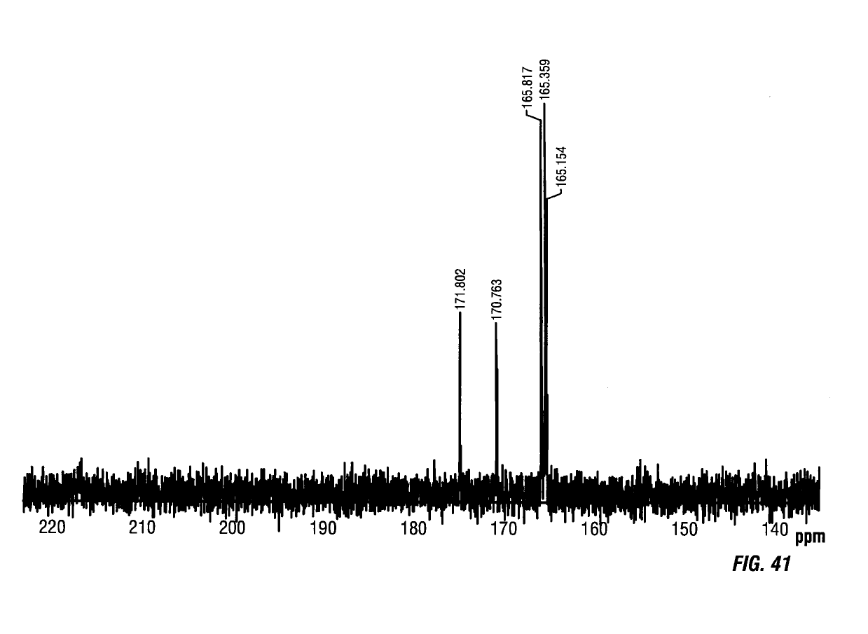

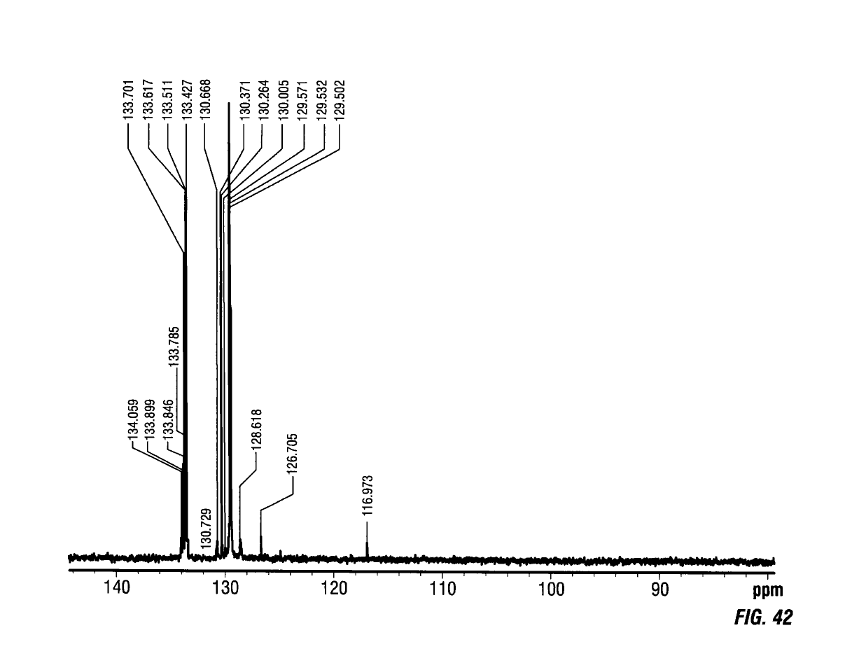

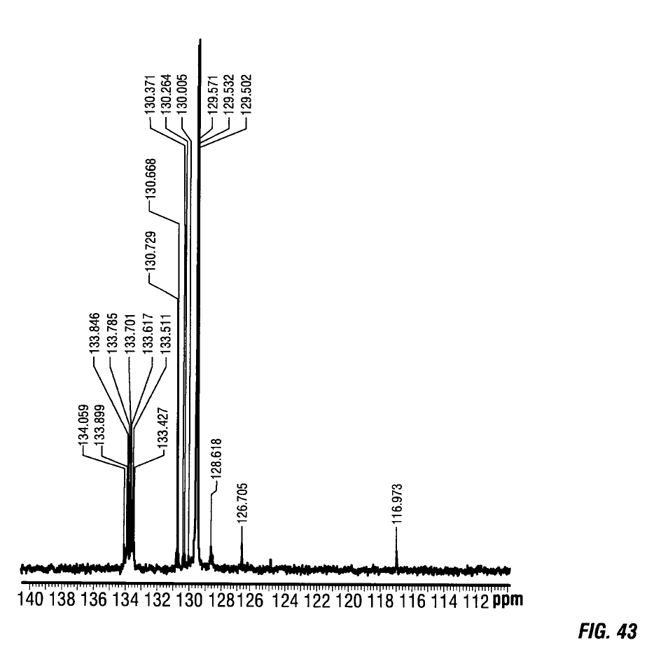

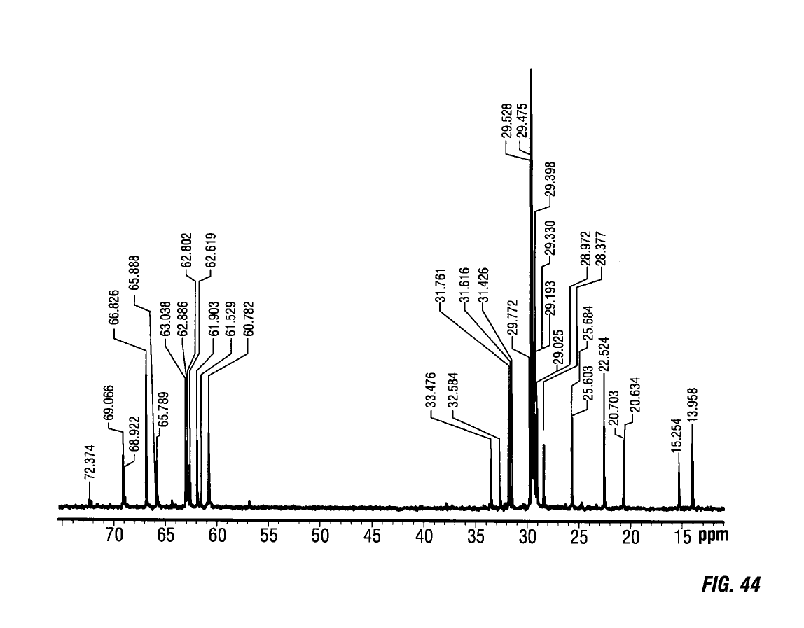

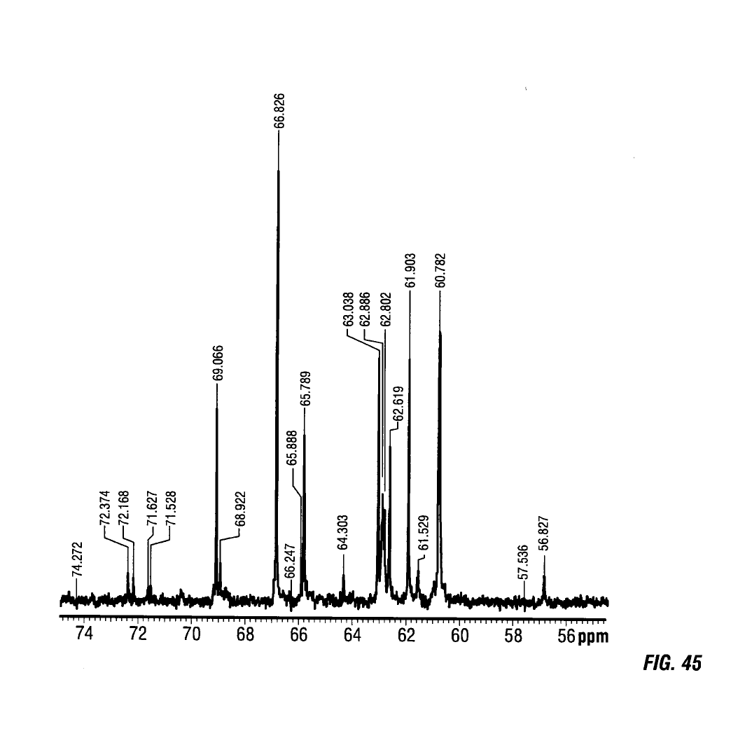

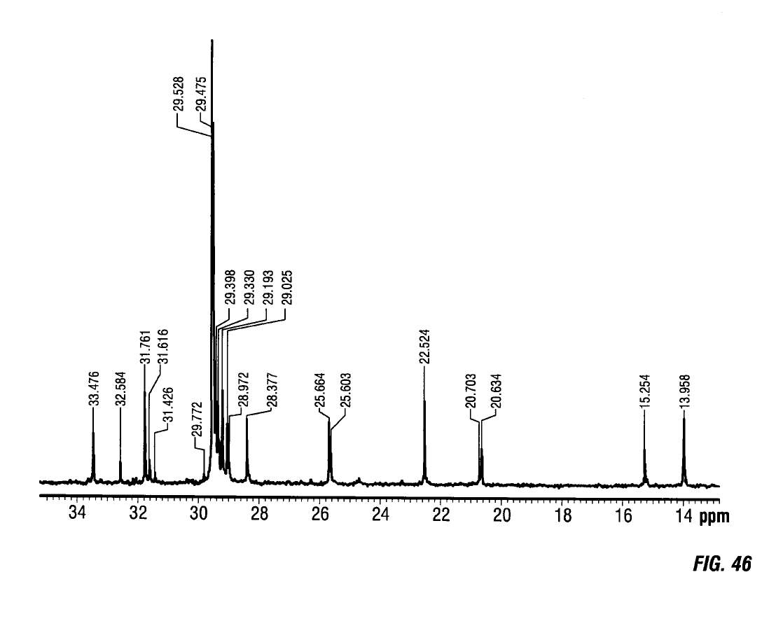

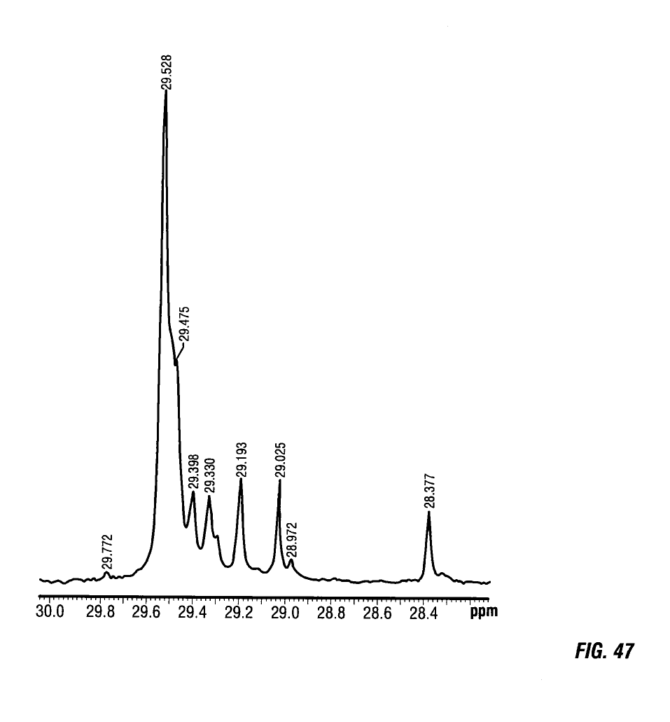

The solubility of the isolated white solid was performed with the following analysis:
Solubility of the isolated white solid
   Mostly soluble: DMSO
   Somewhat soluble: chloroform; methylene chloride
   Insoluble: acetone; acetonitrile; methanol
¹H and ¹³C NMR spectra of the white solid and isolated green filtrate solid; (¹H NMR only of white solid spiked with ethylene glycol, ethylene glycol and DMSO-d6 blank) were obtained and are shown in FIGS. 17-47. White solid melting point: first signs of melting at 81° C.; clear material at 135° C.

Thus, it has been shown that any remaining green tint in the digested rPET can be substantially removed without affecting the properties of the digested rPET.

### dPET Solubility in Organic Solvents

The digestion of recycled PET into discrete molecular weight ranges was developed at Chemir Analytical Services. These materials were all solids and the general trend was higher average molecular weight material was harder than lower average molecular weight material.

All of the materials were very insoluble in common organic solvents as well as water. For instance, the average molecular weights were determined by GPC analyses and sample preparation involved soaking the solids in dichloroacetic acid and chloroform for 1+ hours until it dissolved.

In preparation for exploring commercial uses of rPET, it was necessary to develop a form of dPET which was readily soluble in common organic solvents. Among the ways to do this include:
1. Use of alcohols other than ethylene glycol;
2. Digestion with amines, diamines and polyamines;
3. Combination of ethylene glycol and 2;
4. Reaction of dPET with carboxylic acids or anhydrides to append the hydroxyl end groups.

### Green rPET Treated with Amines and Polyols

Green rPET was treated with various amines and polyols. The reactions were run as previously described: mixtures of green rPET pellets and the amine and/or polyol were heated with zinc acetate. Some reactions included ethylene glycol whiles others did not.

Diamines and hexamethylenetetramines produced hard brittle solids. This was presumably caused by cross-linking between various oligomers/low molecular weight polymers. Although these experiments did not give promising looking material, if indeed cross-linking occurred, the use of limited amounts of diamines/polyamines might prove useful in future experiments as the synthesis of higher molecular weight components from digested PET are targeted.

The reactions run with glycerin and 2,2-dimethyl-1,3-propanediol were promising. A reaction with 1.0 eq 2,2-dimethyl-1,3-propanediol (with respect to monomer MW) and green rPET gave a green pliable material, increasing to 2.0 eq of the diol gave a viscous green syrup. Several other experiments with this diol gave material which ranged from a syrup to a green brittle solid. When glycerin was used, higher amounts of glycerin gave gelatinous material. The results are summarized in Table 22.

TABLE 22

| Notebook | | Ethylene | | | | | |
|---|---|---|---|---|---|---|---|
| ref. | rPET | glycol | Additive 2 | Zn(OAc)₂ | Notes | M_{P} | PI |
| 733-31 | green, | 0.50 eq | 0.19 eq 1,8- | 0.014 eq | Heated to 200-220° C. for 2 h. | 1707 | 1.386 |
| | 310 g | | diaminooctane | | Opaque mint green brittle solid. | | |
| 733-32 | green, | 0.50 eq | 0.42 eq | 0.014 eq | Stirred easily during heat up, | 1060 | 1.233 |
| | 310 g | | ethylenediamine | | reaction solidified at 180° C. | | |
| | | | | | Added 1.0 eq EG and 0.42 eq | | |
| | | | | | ethylenediamine and | | |
| | | | | | temperature increased to | | |
| | | | | | 200° C. Solid remained after | | |
| | | | | | 2 h additional heating. | | |
| | | | | | Presumably, cross-linking | | |
| | | | | | occurred. | | |
| 733-35 | green, | 0.50 eq | 0.27 eq | 0.014 eq | Heated at 200° C. for 52 min | 1015 | 2.602 |
| | 310 g | | hexamethylenete | | to give tar like solution. | | |
| | | | traamine | | Solidified to a dark, hard solid. | | |
| 733-33 | green, | 0.50 eq | 0.33 eq glycerin | 0.014 eq | Heated at 200-210° C. for | 1007 | 1.262 |
| | 310 g | | | | 79 min to form solution. | | |
| | | | | | Mint green waxlike solid. | | |
| 733-34 | green, | 0.50 eq | 0.24 eq 1,6- | 0.014 eq | Heated to 200° C. for 178 | 1301 | 1.303 |
| | 310 g | | hexanediol | | min for a solution to form. | | |
| | | | | | Mint green opaque taffy | | |
| 733-36 | green, | 2.79 eq | — | 0.014 eq | Green rPET digestion with | 672 | 1.113 |
| | 310 g | | | | new rPET lot, standard | | |
| | | | | | stoichiometry. Performed | | |
| | | | | | to demonstrate technique | | |
| | | | | | is reproducible. | | |
| 733-37 | green, | — | 1.0 eq glycerin | 0.014 eq | Heated to 200-225° C. got | 1034 | 1.242 |
| | 310 g | | | | 3 h. Material resembled lime | | |
| | | | | | green jello. | | |
| 733-38 | green, | — | 0.75 eq glycerin | 0.014 eq | Heated to 200° C. for 67 min. | 1030 | 1.303 |
| | 310 g | | | | Material resembled lime | | |
| | | | | | green jello. | | |
| 732-27 | green, | 0.50 eq | 0.3 eq 2,2- | 0.014 eq | Heated at 210° C. for 2 h. | 1132 | 1.242 |
| | 310 g | | dimethyl-1,3- | | Opaque green pliable material, | | |
| | | | propanediol | | resembled Gumby. | | |
| 732-28 | green, | — | 0.6 eq 2,2- | 0.014 eq | Heated at 220° C. for 5.5 h. | 1563 | 1.297 |
| | 310 g | | dimethyl-1,3- | | Clear green sticky pliable | | |
| | | | propanediol | | material, will take and hold | | |
| | | | | | shape when handled. | | |
| 732-29 | green, | — | 1.0 eq 2,2- | 0.014 eq | Heated at 220° C. for 2.5 h. | 1224 | 1.226 |
| | 310 g | | dimethyl-1,3- | | Steady reflux last 78 min | | |
| | | | propanediol | | (ethylene glycol?). Clear green | | |
| | | | | | sticky pliable material, will | | |
| | | | | | take and hold shape when | | |
| | | | | | handled (resembled 732-28). | | |
| 732-30 | green, | — | 2.0 eq 2,2- | 0.014 | Heated to 220° C. for 3.25 h, | 1142 | 1.140 |
| | 155 g | | dimethyl-1,3- | | milky reaction mixture, steady | | |
| | | | propanediol | | reflux. Opaque green liquid, | | |
| | | | | | slightly less viscous than | | |
| | | | | | honey. Glassware easily | | |
| | | | | | cleaned with acetone. | | |
| 732-31 | green, | — | 0.2 eq 2,2- | 0.014 | Heated to 220° C. for 4.5 h, | 3601 | 1.612 |
| | 310 g | | dimethy 1-1,3- | | isolated a dark green brittle | | |
| | | | propanediol | | solid. | | |
| 732-34 | green, | 2.79 eq | — | 0.014 | Heated to 200° C. for 1 h to | 691 | 1.104 |
| | 620 g | | | | give a waxy green solid. | | |
| 733-46 | green, | | 1.5 eq glycerin | 0.014 | Heated to 200-240° C. for | 700 | 1.185 |
| | 310 g | | | | 112 min to give a sticky | | |
| | | | | | green gel. | | |
| 733-48 | green, | — | 0.5 eq 1,6- | 0.014 | Heated to 200° C. for 6.1 h. | 2066 | 1.378 |
| | 310 g | | hexanediol | | Cooled to give a hard mint | | |
| | | | | | green solid. | | |
| 733-49 | dPET | — | 0.5 eq 1,6- | — | dPET (MW 672) was melted | 724 | 1.120 |
| | from | | hexanediol | | at 150° C. and treated with | 1584 | 1.301 |
| | 733-36 | | | | diol. Heated for 3 h, samples | 11439 | 2.211 |
| | | | | | were drawn at t = 0, 1, 2, 3 h | 6647 | 1.936 |
| | | | | | (column to right) and | | |
| | | | | | molecular weight increased. | | |
| | | | | | A flexible green solid was | | |
| | | | | | obtained after cooling. | | |

### Reaction of dPET with Carboxylic Acids and Anhydrides

Predigested material produced in experiment 733-36 from the customary procedure of 2.79 eq ethylene glycol, 1.0 eq rPET and 0.014 eq zinc acetate dihydrate was treated with a variety of diacids and anhydrides in an effort to produce higher molecular weight material. The reactions were performed by heating a mixture of the predigested material along with either a diacid or anhydride. A stream of nitrogen was blown through the flask in to remove water which was produced as a by-product. Oxalic acid was the smallest of the diacids used, and it gave a hard green solid. Higher molecular weight diacids and anhydrides gave materials which ranged from pliable and sticky jello-like material. GPC data is presented for different times in descending order are t=0, t=1 h, t=2 h and t=3 h (or final time point as indicated). The results are summarized in Table 23.

TABLE 23

| Notebk | Predigested | | | | | |
|---|---|---|---|---|---|---|
| ref | PET | Additive | Notes | | MP | PI |
| 733-39 | 1.0 eq | 0.5 eq oxalic acid | Exothermic reaction with considerable outgassing. Heated 3 h at 150° C. Isolated hard green solid. | 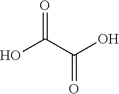 | 676 686 706 729 | 1.121 1.131 1.128 1.130 |
| 733-40 | 1.0 eq | 0.5 eq D-malic acid | Heated 3 h at 150° C. to give a mint green gummy material, very sticky, viscous syrup which could not be separated from aluminum sheet. | 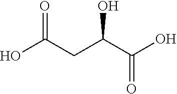 | 697 691 1025 1022 | 1.120 1.149 1.193 1.232 |
| 733-42 | 1.0 eq | 0.5 eq malonic acid | Heated 3 h at 150° C. to give a mint green gummy solid. Material could be hand shaped and hold its form. Sticky, could not be separated from aluminum sheet. | 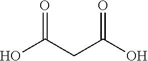 | 687 693 1038 1076 | 1.110 1.146 1.182 1.205 |
| 733-43 | 1.0 eq | 0.5 eq tartaric acid | Heated for 3 h at 150° C. to give a hard green solid. | 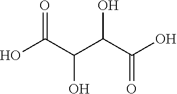 | 707 706 716 673 | 1.121 1.131 1.140 1.171 |
| 733-44 | 1.0 eq | 0.5 eq L-malic acid | Heated for 3 h at 150° C. to give a pliable green solid. Sticky, pliable enough to take and hold a thumb print impression. | 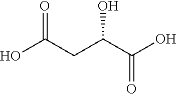 | 659 645 997 1004 | 1.113 1.150 1.191 1.212 |
| 733-45 | 1.0 eq | 0.5 eq maleic anhydride | Heated for 3 h at 150° C. to give a clear green sticky jello-like solid. | 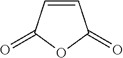 | 670 1006 1372 1646 | 1.107 1.327 1.366 1.432 |
| 733-47 | 1.0 eq | 0.5 eq succinic anhydride | Heated for 3 h at 150° C. to give a dark brittle rubber like solid. | 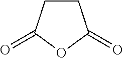 | 680 4336 16165 22674 | 1.122 1.606 2.403 3.575 |
| 733-51 | 1.0 eq | 0.5 eq propionic anhydride | Heated for 2.5 h at 150° C., after 2 h the reaction mixture solidified. Increased temp to 250 C. to give a thick syrup which cooled to a hard mint green solid. | 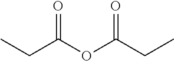 | 791 765 1861 13499 | 1.104 1.122 1.293 2.222 |
| 733-52 | 1.0 eq | 0.5 eq methacrylic anhydride | Heated for 2.5 h at 150° C. A hard brittle green solid was obtained. | 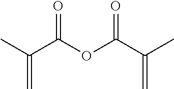 | 766 1375 2098 5248 | 1.143 1.206 1.289 5.433 |
| 733-53 | 1.0 eq | 0.5 eq glutaric anhydride | Heated for 3 h at 150° C. Upon cooling, a gummy mint green mass formed. The material could be deformed and hold a shape. | 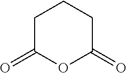 | 828 1230 1242 1593 | 1.096 1.167 1.204 1.260 |
| 733-54 | 1.0 eq | 0.5 eq glutaric acid | Heated for 3 h at 150° C. Upon cooling, a mint green play- dough like mass formed. The material could be deformed and hold a shape, it was harder to manipulate than 733-53. | 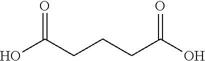 | 842 1246 1581 2366 | 1.106 1.255 1.286 1.334 |
| 733-55 | 1.0 eq | 0.5 eq D-malic acid | dPET was stirred in refluxing toluene and treated with D-malic acid. Refluxed for 3 h with a Dean-Stark trap to collect water. Cooled and collected a gummy off-white solid. (This experiment was similar to 733-40 but with toluene, the previous experiment could not be separated from aluminum foil used in collecting it.) | 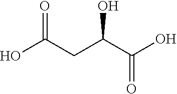 | 829 | 1.107 |
| 733-56 | 1.0 eq | 0.5 eq maleic anhydride | dPET was stirred in refluxing toluene and treated with maleic anhydride. Refluxed for 3 h with a Dean- Stark trap to collect water. Cooled and collected a gummy off-white solid. | 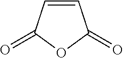 | 852 | 1.117 |
| 733-61 | 1.0 eq | 0.25 eq trimellitic anhydride | dPET was melted at 150° C. and treated with trimellitic anhydride. This was selected to provide cross-linking between oligomers. A sampled at t = 0 when the anhydride was added and at 20 min when the reaction mixture solidified. | 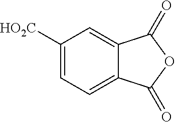 | t = 0: 677 t = 20 min: 2339 | 1.122 2.544 |
| 733-62 | 1.0 eq | 0.25 eq trimellitic anhydride | dPET (732-3, clear MW 672) was stirred in refluxing toluene and treated with trimellitic anhydride for 3 h. Reaction was similar to 733-61 but with solvent to facilitate mixing and removal from reaction flask. Upon cooling to r.t., a hard white solid separated from the toluene layer. | 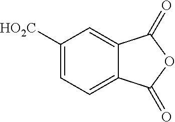 | 701 | 1.117 |
| 733-64 | 1.0 eq | 0.1 pyromellitic anhydride | dPET (732-3, clear MW 672) was stirred in refluxing toluene and treated with pyromellitic anhydride for 3 h. Upon cooling to r.t., a hard white solid separated from the toluene layer. It had the same texture as st. material. | 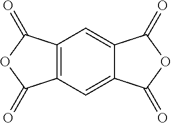 | 714 | 1.139 |

### Solubility Studies

Several of the resulting dPETs were taken up in toluene, NMP, THF and water. dPET from 732-28, 732-29 and 732-30 showed excellent solubility in NMP.

Table 24 describes the results of several digestions of PET (both virgin and recycled) with both neopentyl glycol (NPG) and ethylene glycol (EG) as the glycolysis agents. With respect to the “OH #” column, this refers to the resulting hydroxyl number of the respective dfPET. The appended laboratory notebook pages described in detail the production of the dfPETs listed in Table 24. One of ordinary skill in the art will appreciate that changes can be made to the ratios of reactants, reaction temperature, reaction time, etc. (as is particularly shown in the laboratory notebook pages) to produce dfPETs. Outside of the broad process disclosed herein—one of ordinary skill in the art should not consider any particular combination of reactants and reaction conditions as limiting to the disclosed inventive processes and concept(s) herein.

TABLE 24

| | | | | Molar Ratio | | |
|---|---|---|---|---|---|---|
| | | | | of Alcohol | | |
| | Recycled/ | | | to PET | | |
| Sample | Virgin | | Glycolysis | (monomer | | Avg. |
| Ref. No. | PET | Color | Agent | basis) | OH # | MW |
| 732-29 | recycled | green | NPG | 1.00 | 339 | 1224 |
| 732-34 | recycled | green | EG | 2.78 | 434 | 691 |
| 732-38 | recycled | clear | EG | 2.78 | | 722 |
| 732-40 | recycled | clear | NPG | 1.00 | | 1272 |
| 732-43 | recycled | clear | NPG | 1.00 | 373 | — |
| 732-58 | recycled | green | NPG | 1.00 | 368 | 1158 |
| 732-78 | recycled | green | NPG | 1.00 | 225 | 1268 |
| 732-102 | recycled | green | NPG | 1.00 | | 1198 |
| 733-58 | recycled | clear | NPG | 1.00 | | 1514 |
| 733-65 | recycled | clear | NPG | 1.00 | | 1240 |
| 749-114 | virgin | clear | NPG | 1.00 | 354 | 1237 |
| 749-82 | recycled | green | NPG | 1.00 | | 1410 |
| 751-19 | recycled | green | NPG | 1.00 | 379 | — |
| 753-11 | recycled | green | NPG | 1.00 | 365 | 1246 |
| 768-8 | recycled | green | NPG | 0.40 | 184 | 1920 |

NPG = Neopentyl Glycol
EG = Ethylene Glycol

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of the presently disclosed and claimed inventive concept(s) can be made without departing from its spirit and scope, as will be apparent to those skilled in the art. The specific embodiments described herein are offered by way of example only, and the presently disclosed and claimed inventive concept(s) is to be limited only by the terms of the appended claims, along with the full scope of equivalents to which such claims are entitled.

What is claimed is:

1. An oligomeric form of polyethylene terephthalate having a general formula (I): 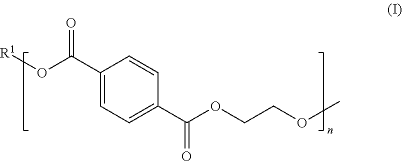
wherein R¹ is a hydrocarbon having at least one functional group selected from the group consisting of a hydroxyl group, an amino group, a carbonyl group and combinations thereof; and
n is an integer of from about 2 to about 500,000.

2. The oligomeric form of polyethylene terephthalate according to claim 1, wherein R¹ is an alkane having at least one functional group selected from the group consisting of a hydroxyl group, an amino group, a carbonyl group and combinations thereof.

3. The oligomeric form of polyethylene terephthalate according to claim 2, wherein R¹ is a linear or branched C₂-C₁₈ alkane having at least one functional group selected from the group consisting of a hydroxyl group, an amino group, a carbonyl group and combinations thereof.

4. The oligomeric form of polyethylene terephthalate according to claim 3, wherein R¹ is a linear or branched C₂-C₈ alkane having at least one functional group selected from the group consisting of at a hydroxyl group, an amino group, a carbonyl group and combinations thereof.

5. An oligomeric form of polyethylene terephthalate comprising having a general formula (II): 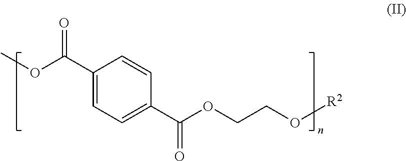
wherein R² is a hydrocarbon having at least one functional group selected from the group consisting of a hydroxyl group, an amino group, a carbonyl group and combinations thereof; and n is an integer of from about 2 to about 500,000.

6. The oligomeric form of polyethylene terephthalate according to claim 5, wherein R² is an alkane having at least one functional group selected from the group consisting of a hydroxyl group, an amino group, a carbonyl group and combinations thereof.

7. The oligomeric form of polyethylene terephthalate according to claim 6, wherein R² is a linear or branched C₂-C₁₈ alkane having at least one functional group selected from the group consisting of a hydroxyl group, an amino group, a carbonyl group and combinations thereof.

8. The oligomeric form of polyethylene terephthalate according to claim 7, wherein R² is a linear or branched C₂-C₈ alkane having at least one functional group selected from the group consisting of at a hydroxyl group, an amino group, a carbonyl group and combinations thereof.

9. An oligomeric form of polyethylene terephthalate comprises a compound represented by a general formula (III): 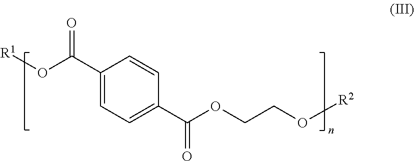
wherein R¹ and R² are identical or different and are each independently a hydrocarbon having at least one functional group selected from the group consisting of a hydroxyl group, an amino group, a carbonyl group and combinations thereof; and n is an integer of from about 2 to about 500,000.

10. The oligomeric form of polyethylene terephthalate according to claim 9, wherein R¹ and R² are alkanes having at least one functional group selected from the group consisting of a hydroxyl group, an amino group, a carbonyl group and combinations thereof.

11. The oligomeric form of polyethylene terephthalate according to claim 10, wherein R¹ and R² are linear or branched C₂-C₁₈ alkanes having at least one functional group selected from the group consisting of a hydroxyl group, an amino group, a carbonyl group and combinations thereof.

12. The oligomeric form of polyethylene terephthalate according to claim 11, wherein R¹ and R² are linear or branched C₂-C₈ alkanes having at least one functional group selected from the group consisting of a hydroxyl group, an amino group, a carbonyl group and combinations thereof.

13. An oligomeric form of polyethylene terephthalate comprising a reaction product of polyethylene terephthalate, a glycolysis agent and a catalyst.

14. The oligomeric form of polyethylene terephthalate according to claim 13, wherein the polyethylene terephthalate is selected from the group consisting of recycled polyethylene terephthalate, virgin polyethylene terephthalate and combinations thereof.

15. The oligomeric form of polyethylene terephthalate according to claim 13, wherein the glycolysis agent is selected from the group consisting of ethylene glycol, diethylene glycol, triethylene glycol, propylene glycol, 1,3-propanediol, 2-methyl-1,3-propanediol, 1,3-butanediol, 1,4-butanediol, 1,5-pentanediol, 1,6-hexanediol, 2,2-diethyl-1,3-propanediol, 1,7-heptanediol, 1,8-ocatanediol, 1,9-nonanediol, 1,4-cyclohexanedimethanol, 2-butyl-2-ethyl-1,3-propanediol, 2-butyl-2-diethyl-1,3-propanediol, 3-methyl-1,5-pentanediol, 2,2,4-trimethylpentanediol, 1,4-dimethyolcyclohexane, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-hetadecanediol, 1,18-octadecanediol, neopentyl glycol, cyclohexanediol, hydrogenated bisphenol A, glycerol, trimethylolpropane, trimethylolethane, diglycerine, triglycerine, pentaerythritol, dipentaerythritol, sorbitol, 1,4-polyisoprenediol, 1,2-polybutadinediol, polybutanediol, and combinations thereof.

16. The oligomeric form of polyethylene terephthalate according to claim 13, wherein the catalyst is zinc acetate.

17. An oligomeric form of polyethylene terephthalate comprising a reaction product of polyethylene terephthalate, a glycolysis agent, an amine and a catalyst.

18. The oligomeric form of polyethylene terephthalate according to claim 17, wherein the polyethylene terephthalate is selected from the group consisting of recycled polyethylene terephthalate, virgin polyethylene terephthalate and combinations thereof

19. The oligomeric form of polyethylene terephthalate according to claim 17, wherein the glycolysis agent is selected from the group consisting of ethylene glycol, diethylene glycol, triethylene glycol, propylene glycol, 1,3-propanediol, 2-methyl-1,3-propanediol, 1,3-butanediol, 1,4-butanediol, 1,5-pentanediol, 1,6-hexanediol, 2,2-diethyl-1,3-propanediol, 1,7-heptanediol, 1,8-ocatanediol, 1,9-nonanediol, 1,4-cyclohexanedimethanol, 2-butyl-2-ethyl-1,3-propanediol, 2-butyl-2-diethyl-1,3-propanediol, 3-methyl-1,5-pentanediol, 2,2,4-trimethylpentanediol, 1,4-dimethyolcyclohexane, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-hetadecanediol, 1,18-octadecanediol, neopentyl glycol, cyclohexanediol, hydrogenated bisphenol A, glycerol, trimethylolpropane, trimethylolethane, diglycerine, triglycerine, pentaerythritol, dipentaerythritol, sorbitol, 1,4-polyisoprenediol, 1,2-polybutadinediol, polybutanediol, and combinations thereof.

20. The oligomeric form of polyethylene terephthalate according to claim 17, wherein the amine is a diamine.

21. The oligomeric form of polyethylene terephthalate according to claim 20, wherein the diamine is selected from the group consisting of ethylene diamine, trimethylene diamine, 1,2-diaminopropane, 1,3-diaminopropane, tetramethylene diamine, pentamethylene diamine, hexamethylene diamine, 1,8-diaminooctane, dodecamethylene diamine, 2,2,4-trimethyl hexamethylene diamine and combinations thereof.

22. The oligomeric form of polyethylene terephthalate according to claim 17, wherein the amine is hexamethylenetetramine.

23. The oligomeric form of polyethylene terephthalate according to claim 17, wherein the catalyst is zinc acetate.

24. An oligomeric form of polyethylene terephthalate comprising a reaction product of polyethylene terephthalate, a glycolysis agent, a diacid and a catalyst.

25. The oligomeric form of polyethylene terephthalate according to claim 24, wherein the polyethylene terephthalate is selected from the group consisting of recycled polyethylene terephthalate, virgin polyethylene terephthalate and combinations thereof

26. The oligomeric form of polyethylene terephthalate according to claim 24, wherein the glycolysis agent is selected from the group consisting of ethylene glycol, diethylene glycol, triethylene glycol, propylene glycol, 1,3-propanediol, 2-methyl-1,3-propanediol, 1,3-butanediol, 1,4-butanediol, 1,5-pentanediol, 1,6-hexanediol, 2,2-diethyl-1,3-propanediol, 1,7-heptanediol, 1,8-ocatanediol, 1,9-nonanediol, 1,4-cyclohexanedimethanol, 2-butyl-2-ethyl-1,3-propanediol, 2-butyl-2-diethyl-1,3-propanediol, 3-methyl-1,5-pentanediol, 2,2,4-trimethylpentanediol, 1,4-dimethyolcyclohexane, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-hetadecanediol, 1,18-octadecanediol, neopentyl glycol, cyclohexanediol, hydrogenated bisphenol A, glycerol, trimethylolpropane, trimethylolethane, diglycerine, triglycerine, pentaerythritol, dipentaerythritol, sorbitol, 1,4-polyisoprenediol, 1,2-polybutadinediol, polybutanediol, and combinations thereof.

27. The oligomeric form of polyethylene terephthalate according to claim 24, wherein the diacid is selected from the group consisting of oxalic acid, malic acid, malonic acid, tartaric acid, glutaric acid, succinic acid, fumaric acid, adipic acid, sebacic acid, maleic acid, azelaic acid, isophthalic acid, terephthalic acid, phthalic acid, terephathalic acid dichloride, 1,4-cyclohexanedicarboxylic acid, 1,3-cyclohexanedicarboxylic acid, naphthalenedicarboxylic acid, 4,4′-biphenyldicarboxylic acid, diphenylmethane-4,4′-dicarboxylic acid, and combinations thereof.

28. The oligomeric form of polyethylene terephthalate according to claim 24, wherein the catalyst is zinc acetate.

29. An oligomeric form of polyethylene terephthalate comprising a reaction product of polyethylene terephthalate, a glycolysis agent, an anhydride and a catalyst.

30. The oligomeric form of polyethylene terephthalate according to claim 29, wherein the polyethylene terephthalate is selected from the group consisting of recycled polyethylene terephthalate, virgin polyethylene terephthalate and combinations thereof.

31. The oligomeric form of polyethylene terephthalate according to claim 29, wherein the glycolysis agent is selected from the group consisting of ethylene glycol, diethylene glycol, triethylene glycol, propylene glycol, 1,3-propanediol, 2-methyl-1,3-propanediol, 1,4-butanediol, 1,5-pentanediol, 1,6-hexanediol, 2,2-diethyl-1,3-propanediol, 1,7-heptanediol, 1,8-ocatanediol, 1,9-nonanediol, 1,4-cyclohexanedimethanol, 2-butyl-2-ethyl-1,3-propanediol, 2-butyl-2-diethyl-1,3-propanediol, 3-methyl-1,5-pentanediol, 2,2,4-trimethylpentanediol, 1,4-dimethyolcyclohexane, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-hetadecanediol, 1,18-octadecanediol, neopentyl glycol, hydrogenated bisphenol A, glycerol, trimethylolpropane, trimethylolethane, diglycerine, triglycerine, pentaerythritol, dipentaerythritol, sorbitol, 1,4-polyisoprenediol, 1,2-polybutadinediol, polybutanediol, and combinations thereof.

32. The oligomeric form of polyethylene terephthalate according to claim 29, wherein the anhydride is selected from the group consisting of propionic anhydride, maleic anhydride, succinic anhydride, methacrylic anhydride, glutaric anhydride, trimellitic anhydride, pyromellitic anhydride, phthalic anhydride, tetrabromophthalic anhydride, tetrachlorophthalic anhydride, hexahydrophthalic anhydride, tetrahydrophthalic anhydride, and combinations thereof.

33. The oligomeric form of polyethylene terephthalate according to claim 29, wherein the catalyst is zinc acetate.
